(19) United States
(12) Patent Application Publication
(10) Pub. No.: US 2025/0327800 A1
(43) Pub. Date: Oct. 23, 2025

(54) COMPOSITIONS AND METHODS FOR DETECTING LYMPHATIC FILARIASIS

(71) Applicant: The United States of America, as represented by the Secretary, Dept. of Health and Human Services, Bethesda, MD (US)

(72) Inventors: Thomas B. Nutman, Chevy Chase, MD (US); Sasisekhar Bennuru, Clarksburg, MD (US)

(73) Assignee: The United States of America, as represented by the Secretary, Dept. of Health and Human Services, Bethesda, MD (US)

(21) Appl. No.: 18/869,777

(22) PCT Filed: May 31, 2023
(86) PCT No.: PCT/US2023/067703

§ 371 Date: Nov. 26, 2024

Related U.S. Application Data
(60) Provisional application No. 63/347,794, filed on Jun. 1, 2022.

(51) Int. Cl. G01N 33/569; C07K 14/435

(52) U.S. Cl. CPC G01N 33/56905; C07K 14/4354; C07K 2319/61; G01N 2333/4353; G01N 2469/20

(57) ABSTRACT

Compositions for detecting agents causing lymphatic filariasis, including Wuchereria bancrofti, are provided, including the antigen Wb5 or fusion proteins including Wb5 linked to a reporter protein or tag. Nucleic acids encoding the Wb5 protein or the fusion proteins, vectors including the nucleic acids, and host cells including the nucleic acids or vectors are also provided. Methods of detection of antibodies to Wb5 in a sample from a subject, including immunoassay methods are also provided.

### CROSS REFERENCE TO RELATED APPLICATIONS

This application claims priority to U.S. Provisional Application No. 63/347,794, filed Jun. 1, 2022, which is incorporated by reference in its entirety.

### FIELD

This disclosure relates to compositions and methods for detecting lymphatic filariasis, particularly Wuchereria bancrofti.

### BACKGROUND

Lymphatic filariasis (LF) is a neglected tropical disease characterized by lymphedema, primarily in the legs. In addition, the swelling and decreased lymph system function makes affected individuals more susceptible to bacterial infections of the skin and lymph system, leading to hardening and thickening of the skin, which is referred to as elephantiasis. LF affects over 120 million people throughout the tropics and sub-tropics of Asia, Africa, the Western Pacific, and parts of the Caribbean and South America.

Larvae (L3) of the parasitic roundworms Wuchereria bancrofti or Brugia malayi are transmitted to the host by infected mosquitos. The infective L3 larvae migrate from the skin to the lymphatic vessels, where they mature into adults. It is now understood that infection typically occurs during childhood, with a long incubation of subclinical disease prior to clinical symptoms manifesting during adulthood. Treatment options are limited, and current chemotherapeutic options have limited effects against adult worms. Preventive programs use mass drug administration to eliminate microfilariae from the community, disrupting transmission by mosquitos.

Current methods of confirming active infection by W. bancrofti or B. malayi include microscopy and immunoassays using serum from individuals. The sensitivity of microscopic detection can vary between patients, and in some instances may depend on the time of day of collection of the serum sample. Immunoassays are generally considered more sensitive and the serum can be collected at any time. Current immunoassays test for circulating filarial antigen, a 200 kilodalton protein that is specific to W. bancrofti. However, this antigen shows cross-reactivity with antibodies directed towards other parasites, such as B. malayi, Loa loa, or Onchocerca volvulus, whose geographic distribution often overlaps with that of W. bancrofti.

To combat the social and economic costs of LF, the World Health Organization established the Global Programme to Eliminate Lymphatic Filariasis (GPELF) in 2000. Mass drug administration (MDA) has been utilized in areas where prevalence is high, and as of 2020, 48 of the 72 countries where W. bancrofti and B. malayi are endemic still required MDA to control the spread of LF parasites, affecting over 850 million people. The ability to accurately detect recent exposure is critical to surveillance and MDA efforts. Thus, there remains a need for specific and sensitive assays for detection of W. bancrofti infection.

### SUMMARY

Disclosed herein is an antigen from W. bancrofti or B. malayi that can be used for specific and sensitive detection of one or more agents causing lymphatic filariasis, such as W. bancrofti or Brugia sp. In some aspects, the antigen is designated Wb5 and includes a protein with at least 90% sequence identity to the amino acid sequence of any one of SEQ ID NO: 2, SEQ ID NO: 4, SEQ ID NO: 8, or SEQ ID NO: 10, or includes or consists of the amino acid sequence of any one of SEQ ID NO: 2, SEQ ID NO: 4, SEQ ID NO: 8, or SEQ ID NO: 10. In other aspects, the antigen is a portion of a Wb5 protein and includes a peptide with at least 90% sequence identity to the amino acid sequence of any one of SEQ ID NOs: 18-21 or includes or consists of the amino acid sequence of any one of SEQ ID NOs: 18-21.

Provided herein are fusion proteins that include a Wb5 protein or a portion thereof linked to at least one reporter protein or at least one tag. In some aspects, the Wb5 protein includes an amino acid sequence with at least 90% sequence identity to the amino acid sequence of any one of SEQ ID NO: 2, SEQ ID NO: 4, SEQ ID NO: 8, or SEQ ID NO: 10, or a portion thereof, or includes or consists of the amino acid sequence of any one of SEQ ID NO: 2, SEQ ID NO: 4, SEQ ID NO: 8, SEQ ID NO: 10, or a portion thereof. In other aspects, the Wb5 protein or portion thereof includes an amino acid sequence with at least 90% sequence identity to the amino acid sequence of any one of SEQ ID NOs: 18-21 or includes or consists of the amino acid sequence of any one of SEQ ID NOs: 18-21.

In specific examples, the reporter protein is a luciferase protein, such as Renilla luciferase. In other examples, the tag is a 6× histidine tag, glutathione-S-transferase (GST), IgG-Fc, maltose-binding protein (MBP), or biotin. In some examples, the fusion protein includes an amino acid sequence with at least 90% sequence identity to the amino acid sequence of SEQ ID NO: 14 or SEQ ID NO: 16 or includes or consists of the amino acid sequence of SEQ ID NO: 14 or SEQ ID NO: 16.

Also provided are nucleic acids that encode the disclosed fusion proteins. In some examples, the nucleic acid has at least 90% sequence identity to the nucleic acid sequence of SEQ ID NO: 15 or SEQ ID NO: 17. In other examples, the nucleic acid includes or consists of the nucleic acid sequence of SEQ ID NO: 15 or SEQ ID NO: 17.

In additional aspects, a codon-optimized nucleic acid encoding a Wb5 protein or a portion thereof is provided. In some aspects, the nucleic acid encodes an amino acid sequence with at least 90% sequence identity to the amino acid sequence of SEQ ID NO: 2 or a portion thereof, or encodes a protein that includes or consists of the amino acid sequence of SEQ ID NO: 2 or a portion thereof. In some examples, the nucleic acid has at least 90% sequence identity to the nucleic acid sequence of SEQ ID NO: 1 or includes or consists of SEQ ID NO: 1, or a portion thereof. In other aspects, the nucleic acid encodes an amino acid sequence with at least 90% sequence identity to the amino acid sequence of SEQ ID NO: 4 or a portion thereof, or encodes a protein that includes or consists of the amino acid sequence of SEQ ID NO: 4 or a portion thereof. In some examples, the nucleic acid has at least 90% sequence identity to the nucleic acid sequence of SEQ ID NO: 3 or includes or consists of SEQ ID NO: 3, or a portion thereof. In additional aspects, the nucleic acid has at least 90% sequence identity to the nucleic acid sequence of any one of SEQ ID NOs: 11-13, or includes or consists of the nucleic acid sequence of any one of SEQ ID NOs: 11-13. In other examples, the nucleic acid encodes a portion of a Wb5 protein with at least 90% sequence identity to the amino acid sequence of any one of SEQ ID NOs: 18-21 or encodes a portion of a Wb5 protein including or consisting of the amino acid sequence of any one of SEQ ID NOs: 18-21.

Also provided are vectors that include a nucleic acid encoding a disclosed fusion protein, a disclosed codon-optimized nucleic acid encoding a Wb5 protein or portion thereof (such as any one of SEQ ID NOs: 1, 3, or 11-13), or a nucleic acid with at least 90% sequence identity to the nucleic acid sequence of any one of SEQ ID NOs: 5-7 or 9 or a nucleic acid including or consisting of any one of SEQ ID NOs: 5-7 or 9, or a portion thereof. In additional aspects, a host cell including a disclosed nucleic acid or vector is provided. In some examples, the host cell is a bacterial, insect, or mammalian host cell. In one example, the host cell is an HEK293 cell.

Also provided are methods of detecting presence of antibodies to W. bancrofti or Brugia sp., such as antibodies to W. bancrofti or Brugia sp. Wb5, in a sample. In some aspects, the methods include contacting the sample with a disclosed fusion protein including a reporter protein under conditions sufficient to form a complex between the Wb5 protein or a portion thereof and an antibody to W. bancrofti or Brugia sp.; contacting the complex with an immobilized binding agent capable of binding to the antibody to WE. bancrofti or Brugia sp., thereby forming an immobilized complex including the fusion protein; and detecting output from the reporter protein in the immobilized complex, thereby detecting presence of antibodies to W. bancrofti or Brugia sp. in the sample. In some examples, the immobilized binding agent is protein A, protein G, or protein A/G. In some examples, the reporter protein is luciferase and the method includes contacting the immobilized complex with a luciferase enzyme substrate (such as coelenterazine).

In other aspects, methods of detecting presence of antibodies to W. bancrofti or Brugia sp., such as antibodies to W. bancrofti or Brugia sp. Wb5, in a sample using an immunoassay are provided. In some aspects, the immunoassay is an ELISA assay, a bead-based antibody, or a lateral flow assay. In one aspect, a Wb5 protein or portion thereof or a fusion protein including a Wb5 protein or portion thereof is attached to a solid support. In some examples, the Wb5 protein or portion thereof attached to the solid support is contacted with a sample from a subject to form a first complex including the Wb5 protein or portion thereof an antibody to Wb5. The complex is contacted with a secondary antibody that includes a detectable label to form a second complex including the first complex and the secondary antibody. Presence of the second complex is detected by detecting output from the detectable label. In some examples, the secondary antibody is an anti-human IgG antibody, such as an anti-human IgG4 antibody. The detectable label may include an enzyme (such as horseradish peroxidase or alkaline phosphatase).

In some aspects, the disclosed methods further include detecting presence of antibodies to W. bancrofti Wb123 protein in the sample.

In some aspects, the sample is from at least one subject infected with or suspected to be infected with W. bancrofti or Brugia sp. In some examples, the sample is from a single subject. In other examples, the sample is a pooled sample from a plurality of subjects. In some aspects, the sample is blood, serum, or plasma. In some aspects, the sample is from a single subject, and the subject is diagnosed with lymphatic filariasis when presence of antibodies to W. bancrofti Wb5 protein are detected in the sample from the subject. In some examples, the methods further comprise treating the subject for lymphatic filariasis. In other aspects, the sample is a pooled sample, and one or more lymphatic filariasis control regimens is selected when presence of antibodies to W. bancrofti Wb5 protein are detected in the pooled sample. The methods may further comprise implementing the one or more lymphatic filariasis control regimens in a population from which the pooled sample was obtained.

Also provided are kits for detecting antibodies to W. bancrofti or Brugia sp., such as antibodies to Wb5 protein. In some aspects, the kits include a disclosed Wb5 fusion protein and an immobilized binding agent capable of binding to antibodies to Wb5 protein (such as protein A, protein G, or protein A/G). In other aspects, the kits include a Wb5 protein or portion thereof linked to a substrate, such as a lateral flow test strip or a multiwell plate.

The foregoing and other features of the disclosure will become more apparent from the following detailed description, which proceeds with reference to the accompanying figures.

### BRIEF DESCRIPTION OF THE DRAWINGS

FIGS. 1A-1G are graphs showing signal to noise ratio of the indicated proteins for total IgG from pooled sera. Wb-India MF+, Wb-CI-Pool1, and WB-Pool2: pooled sera from Wb-infected mf-positive individuals; WB-CP: Wb-chronic pathology (lymphedema); Wb-EN: endemic normals from India; BBN and NEN: healthy blood bank normals; Loa: Loa loa-infected; OvCameroon, OvEC, OvGU: Onchocerca-infected from Cameroon, Ecuador, and Guatemala, respectively.

FIGS. 2A and 2B show IgG and IgG4 seroreactivity for Wb5A (FIG. 2A) and Wb5B (FIG. 2B) in pooled sera (labeling as in FIGS. 1A-1G). Note difference in y-axis scale between FIGS. 2A and 2B.

FIGS. 3A-3D showWb5A (FIG. 3A) and Wb5B (FIG. 3B) specificity in individual serum samples. MF: Wb-infected mf-positive; PRE: infected mf-positive individuals from Cook Islands in 1974; POST: longitudinally followed up samples of individuals from Cook Islands that were treated and mf-negative and circulating antigen negative in 1992 (after 18 years); CP: Wb-chronic pathology (lymphedema); LOA: Loa loa-infected; OV: Onchocerca-infected; STRONGY: Strongyloides-infected; EN and BB: healthy blood bank normals; Jird_Pre: naïve gerbil sera before infection with B. malayi L3 larvae; Jird_Necr: infected gerbil sera obtained during necropsy 3 months post-infection FIGS. 3C and 3D represent the paired data for the Cook Island samples from 1974 (Pre) and 1992 (Post) for Wb5A and Wb5B, respectively. Note differences in y-axis scale between FIGS. 3A and 3B and FIGS. 3C and 3D.

FIG. 4 is a schematic diagram of an exemplary lateral flow assay to detect Wb5 in a sample.

FIG. 5 is a graph showing correlation of IgG4 reactivity to Wb5 between LIPS and Luminex-based assays. The ability of Wb5 to detect W. bancrofti microfilaria positive samples is highly correlated in LIPS and Luminex platforms. Detection by Wb5 of a subset of W. bancrofti microfilaria positive samples was tested in both LIPS and Luminex IgG4-based systems (n=85). Detection intensity was highly linearly correlated (R²=0.9575).

FIG. 6 is a graph showing IgG4 reactivity to Wb5 in Luminex-based assay. Wb5 and Wbl23 reactivity with individual sera from microfilaria positive individuals infected with W. bancrofti microfilaria positive (MF+), B. malayi (BM), Other helminths (O. volvulus, L. loa, S. stercoralis, M. perstans), or healthy blood bank and endemic normal (Helminth uninfected).

FIG. 7 is a heatmap depicting Wb5 and Wb123 positive and negative results from IgG4 Luminex testing.

FIG. 8 is a graph showing LIPS screening of IgG anti-Wb5 and anti-Wb123 antibodies in one patient over a 22-year time period following definitive filarial treatment. Data plotted as relative light units (RLU).

FIG. 9 is a graph showing screening of overlapping peptides of Wb5 with pooled infected sera.

FIG. 10 shows the IgG4 reactivity to Wb5 in Luminex based assay with individuals infected with Brugia spp. The dark gray (B. malayi) and light gray (B. timori) dots represent archived samples from two different regions of the world.

### SEQUENCE LISTING

Any nucleic acid and amino acid sequences provided herein or in the accompanying Sequence Listing are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand.

SEQ ID NO: 1 is an exemplary codon-optimized Wb5B encoding nucleic acid sequence:
GGATCCATGCGGTCCGCCCAGTTCCCCTTCTTTATCTCTCCTCTGCTGTTCTTTATCATCGGCACCCTGGCC
CTGCTGGCCATGTCTCAGAGGTGCGCCCCTAGCATCAACAAGGACAACCTGAATCACGAGGATGGCGACGAT
GGCAACATCAACAATAACGGCGACAATAACATCAATGGCGACGATAACAACATCAACAACAACATCAACGAC
AACAATAACTTCCTGCAGCAGCAGCGGTACGATGAGGCCCTGAGCATCGAGGGCCTGTCCTGGGACGATATC
ACCGAGGAGGAGAGGGATATCCTGATGAGCCTGCTGCTGAACCGCTATATCAATGCCTCCATGCTGCCATGG
AATAACAATGGCATCCCCGTGGTGGTGAACGTGATCCGGTCTGCCCTGCCCCACAATAGAGGCCAGTTTATC
GGCTACACAGGCCTGCTGGAGCTGTGACTCGAG
SEQ ID NO: 2 is an exemplary Wb5B amino acid sequence:
QRCAPSINKDNLNHEDGDDGNINNNGDNNINGDDNNINNNINDNNNELQQQRYDEALSIEGLSWDDITEEER
DILMSLLLNRYINASMLPWNNNGIPVVVNVIRSALPHNRGQFIGYTGLLEL
SEQ ID NO: 3 is an exemplary codon-optimized Wb5A encoding nucleic acid sequence:
ATGCGGTCCGCCCAGTTCCCCTTCTTTATCTCTCCTCTGCTGTTCTTTATCATCGGCACCCTGGCCCTGCTG
GCCATGTCTCAGAGGTGCGCCCCTAGCATCAACAAGGACAACCTGAATCACGAGGATGGCGACGATGGCAAC
ATCAACAATAACGGCGACAATAACATCAATGGCGACGATAACAACATCAACAACAACATCAACGACAACAAT
AACTTCCTGCAGCAGCAGCGGTACGATGAGGCCCTGAGCATCGAGGGCCTGTCCTGGGACGATATCACCGAG
GAGGAGAGGGATATCCTGATGAGCCTGCTGCTGAACCGCTATATCAATGCCTCCATGCTGCCATGGAATAAC
AATGGCATCCCCGTGGTGGTGAACGTGATCCGGTCTGCCCTGCCCCACAATAGAGGCCAGTTTATCGGCTAC
ACAGGCCTGCTGGAGCTG
SEQ ID NO: 4 is an exemplary Wb5A amino acid sequence:
MRSAQFPFFISPLLFFIIGTLALLAMSQRCAPSINKDNLNHEDGDDGNINNNGDNNINGDDNNINNNINDNN
NFLQQQRYDEALSIEGLSWDDITEEERDILMSLLLNRYINASMLPWNNNGIPVVVNVIRSALPHNRGQFIGY
TGLLEL
SEQ ID NO: 5 is a native W. bancrofti Wb5 protein predicted coding sequence
(PRJEB536):
ATGAGAAGCGCACAATTTCCATTCTTCATATCACCATTACTTTTTTTCATTATCGGTACACTAGCATTATTA
GCAATGTCACAAAGATGTGCTCCGAGCATTAACAAAGACAACTTGAACCATGAAGATGGTGATGACGGTAAC
ATAAACAATAATGGTGACAATAATATTAATGGTGACGACAACAACATTAACAACAACATTAATGACAACAAT
AATTTTCTCCAGCAACAAAGATATGATGAAGCTCTTTCCATTGAAGGCTTATCTTGGGATGATATCACGGAA
GAGGAAAGAGATATTCTCATGTCATTGCTACTTAACCGTTATATTAATGCATCAATGTTACCGTGGAATAAT
AATGGTATACCTGTTGTGGTCAATGTTATTAGAAGTGCCTTACCTCATAATCGTGGTCAATTTATCGGCTAT
ACTGGCCTGTTGGAACTT
SEQ ID NO: 6 is a native W. bancrofti Wb5 protein predicted coding sequence
(PRJNA275548):
ATGAGAAGCGCACAATTTCCATTCTTCATATCACCATTACTTTTTTTCATTATCGGTACACTAGCATTATTA
GCAATGTCACAAAGATTAATAATGGTATACCTGTTGTGGTCAATGTTATTAGAAGTGCCTTACCTCATAATC
GTGGTCAATTTATCGGCTATACTGGCCTGTTGGAACTTTAAGCTTATTTATGCGCACATATTAATACCA
SEQ ID NO: 7 is a native W. bancrofti Wb5 protein predicted coding sequence:
TTAAGGTATCGAATTCCGGAACGATGAGAAGCGCACAATTTCCATTCTTCATATCACCATTACTTTTTTTCA
TTATCGGTACACTAGCATTATTAGCAATGTCACAAAGATGTGCTCCGAGCATTAACAAAGACAACTTGAACC
ATGAAGATGGTGATGACGGTAACATAAACAATAATGGTGACAATAATATTAATGGTGACGACAACAACATTA
ACAACAACATTAATGACAACAATAATTTTCTCCAGCAACAAAGATATGATGAAGCTCTTTCCATTGAAGGCT
TATCTTGGGATGATATCACGGAAGAGGAAAGAGATATTCTCATGTCATTGCTACTTAACCGITATATTAATG
CATCAATGTTACCGTGGAATAATAATGGTATACCTGTTGTGGTCAATGTTATTAGAAGTGCCTTACCTCATA
ATCGTGGTCAATTTATCGGCTATACTGGCCTGTTGGAACTTTAAGCTTATTTATGCGCACATATTAATACCA
TAAAATTTGGTGATATTTATTGTTGAATAAAATTGAAAAACTGAAA
SEQ ID NO: 8 is an exemplary native W. bancrofti Wb5 protein sequence (PRJNA275548):
MRSAQFPFFISPLLFFIIGTLALLAMSQRLIMVYLLWSMLLEVPYLIIVVNLSAILACWNEKLIYAHILIP
SEQ ID NO: 9 is a native B. malayi Wb5 protein predicted coding sequence
(PRJNA10729):
ATGAGAAGCGCACAATTTCCACTTTTCATATCACCATTATCTTTTTGCATTATCGGTGCACTAGCATTACTA
GCAATGTCGCAAAGATGTGCTCCGAGCATTAACAGAAATAACTTAAATCATGAAGATGGTGATGACGGTAAC
ATAAACGATAATGGTGACAATAATGTTAATGGTGACGATGGCAATATCAACAACGTTAATGACAACAATAAT
TTTCTCCAGCAGCAAAGATATGATGAAGCTCTTTCCATTGAAGGCTTATCTTGGGATAATATTACGGAAGAG
GAGAGAGATATTCTTATGTCATTGCTACTTAACCGATATATTAATGCATCAATGCTACCGTGGAATAATGAT
GGTATACCTGTTGTGGTCAATGTTATTAGAAGTGCTTTACCTCGCAATTATGCCAATTTATCGGCTATACTG
GCCTATTGGAGCAT
SEQ ID NO: 10 is a B. malayi Wb5 protein sequence (PRJNA10729):
MRSAQFPLFISPLSFCIIGALALLAMSQRCAPSINRNNLNHEDGDDGNINDNGDNNVNGDDGNINNVNDNNN
FLQQQRYDEALSIEGLSWDNITEEERDILMSLLLNRYINASMLPWNNDGIPVVVNVIRSALPRNYGQFIGYT
GLLEH
SEQ ID NO: 11 is an exemplary Wb5A encoding nucleic acid sequence codon-optimized
for expression in mammalian cells (including 6xHis tag):
ATGCGAAGTGCACAATTTCCATTTTTTATCTCTCCTCTGCTGTTCTTCATCATCGGCACCCTGGCTCAGAGA
TGCGCCCCATCTATCAACAAGGACAACCTGAACCACGAGGACGGCGATGATGGCAACATCAACAACAACGGA
GACAATAATATTAATGGAGACGACAACAACATCAACAACAACATCAACGACAACAACAACTTCCTGCAGCAG
CAACGCTACGACGAGGCCCTGTCCATCGAGGGCCTGTCTTGGGACGATATCACCGAGGAAGAGCGGGACATC
CTGATGTCCCTGCTGCTGAACAGATACATCAACGCCTCCATGCTGCCTTGGAACAATAACGGCATCCCCGTC
GTGGTGAACGTGATCAGAAGCGCTCTGCCTCACAACCGGGGCCAGITTATCGGCTACACCGGCCTCCTGGAA
CTGCACCACCATCACCACCAC
SEQ ID NO: 12 is an exemplary Wb5A encoding nucleic acid sequence codon-optimized
for expression in E. coli:
ATGCAAAGGTGTGCTCCCTCAATAAATAAAGACAACCTGAATCATGAGGATGGCGACGACGGCAATATCAAC
AACAACGGCGACAACAACATCAATGGCGATGATAACAACATCAACAACAACATCAATGATAATAACAACTTC
CTGCAGCAACAACGTTACGACGAGGCGCTGTCTATTGAAGGTCTCTCCTGGGATGACATCACCGAAGAGGAG
CGCGATATCCTGATGAGCCTGTTATTGAATCGCTACATTAACGCTAGCATGCTGCCGTGGAATAACAACGGT
ATTCCGGTGGTTGTTAATGTGATTCGTAGCGCACTGCCGCATAATCGTGGTCAGTTTATTGGTTATACGGGT
TTGTTGGAACTG
SEQ ID NO: 13 is an exemplary Wb5A encoding nucleic acid sequence codon-optimized
for expression in insect cells (with 6xHis tag):
GCCGCCACCATGCAGCGGTGCGCACCTTCGATAAACAAAGATAACCTGAATCATGAGGATGGTGATGACGGG
AACATAAACAATAACGGCGACAATAACATAAATGGCGACGACAACAATATCAACAACAATATCAACGACAAT
AATAACTTTTTGCAGCAACAACGTTACGATGAAGCCCTTAGTATTGAGGGGCTATCCTGGGATGACATTACC
GAAGAAGAGAGGGATATACTAATGTCATTATTGCTCAACCGCTATATTAATGCGAGCATGCTGCCATGGAAT
AATAACGGCATCCCCGTAGTCGTTAATGTGATTCGATCTGCTTTACCGCATAATAGAGGACAGTTCATCGGT
TATACTGGACTCCTTGAATTACATCACCATCACCACCACTGA
SEQ ID NO: 14 is the amino acid sequence of a Wb5 fusion protein with cleavable 6xHis
tag (bold, Wb5 signal sequence; underlined, 6xHis tag; italics, TEV protease cleavage
site; bold underlined, Wb5B protein):
MRSAQFPFFISPLLFFIIGTLAHHHHHHENLYFQGQRCAPSINKDNLNHEDGDDGNINNNGDNNINGDDNNI
NNNINDNNNFLQQQRYDEALSIEGLSWDDITEEERDILMSLLLNRYINASMLPWNNNGIPVVVNVIRSALPH
NRGQFIGYTGLLEL
SEQ ID NO: 15 is an exemplary nucleic acid encoding a Wb5 fusion protein with cleavable
6xHis tag, codon-optimized for expression in mammalian cells (bold, Wb5 signal sequence;
underlined, 6xHis tag; italics, TEV protease cleavage site; bold underlined, Wb5B):
ATGCGAAGTGCACAATTTCCATTTTTTATCTCTCCTCTGCTGTTCTTCATCATCGGCACCCTGGCTCACCAC
CACCACCACCACGAGAACCTGTACTTCCAGGGCCAGAGATGCGCCCCATCTATCAACAAGGACAACCTGAAC
CACGAGGACGGCGATGATGGCAACATCAACAACAACGGAGACAATAATATTAATGGAGACGACAACAACATC
AACAACAACATCAACGACAACAACAACTTCCTGCAGCAGCAACGCTACGACGAGGCCCTGTCCATCGAGGGC
CTGTCTTGGGACGATATCACCGAGGAAGAGCGGGACATCCTGATGTCCCTGCTGCTGAACAGATACATCAAC
GCCTCCATGCTGCCTTGGAACAATAACGGCATCCCCGTCGTGGTGAACGTGATCAGAAGCGCTCTGCCTCAC
AACCGGGGCCAGTTTATCGGCTACACCGGCCTCCTGGAACTG
SEQ ID NO: 16 is the amino acid sequence of an exemplary Wb5-Fc fusion protein amino
acid sequence with Fc tag (bold, Wb5 signal sequence; italics hIgG1-Fc fragment;
underlined, linker; bold italics, FLAG-tag; bold underlined, Wb5B protein):
MRSAQFPFFISPLLFFIIGTLADKTHTCPPCPAPELLGGPSVFLFPPKPKDTLMISRTPEVTCVVVDVSHED
PEVKFNWYVDGVEVHNAKIKPREEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYKTTPPVLDSDGSFFLYSKLTVDK
SRWQQGNVFSCSVMHEALHNHYTQKSLSLSPGKAAANSSIDLISVPVDSRRPACKIPNDLKQKVMNHDYKDD
DDKRCAPSINKDNLNHEDGDDGNINNNGDNNINGDDNNINNNINDNNNFLQQQRYDEALSIEGLSWDDITEE
ERDILMSLLLNRYINASMLPWNNNGIPVVVNVIRSALPHNRGQFIGYTGLLEL
SEQ ID NO: 17 is a nucleic acid sequence encoding an exemplary Wb5-Fc fusion protein,
codon-optimized for expression in mammalian cells (bold, Wb5 signal sequence; italics
hIgG1-Fc fragment; underlined, linker; bold italics, FLAG-tag; bold underlined,
Wb5B):
ATGCGAAGTGCACAATTTCCATTTTTTATCTCTCCTCTGCTGTTCTTCATCATCGGCACCCTGGCTGACAAG
ACCCACACCTGCCCCCCCTGCCCCGCCCCCGAGCTGCTGGGCGGCCCCTCCGTGTTCCTGTTCCCCCCCAAG
CCCAAGGACACCCTGATGATCTCCAGGACCCCCGAGGTGACCTGCGTGGTGGTGGACGTGTCCCACGAGGAC
CCCGAGGTGAAGTTCAACTGGTACGTGGACGGCGTGGAGGTGCACAACGCCAAGACCAAGCCCAGGGAGGAG
CAGTACAACTCCACCTACAGGGTGGTGTCCGTGCTGACCGTGCTGCACCAGGACTGGCTGAACGGCAAGGAG
TACAAGTGCAAGGTGTCCAACAAGGCCCTGCCCGCCCCCATCGAGAAGACCATCTCCAAGGCCAAGGGCCAG
CCCAGGGAGCCCCAGGTGTACACCCTGCCCCCCTCCAGGGAGGAGATGACCAAGAACCAGGTGTCCCTGACC
TGCCTGGTGAAGGGCTTCTACCCCTCCGACATCGCCGTGGAGTGGGAGTCCAACGGCCAGCCCGAGAACAAC
TACAAGACCACCCCCCCCGTGCTGGACTCCGACGGCTCCTTCTTCCTGTACTCCAAGCTGACCGTGGACAAG
TCCAGGTGGCAGCAGGGCAACGTGTTCTCCTGCTCCGTGATGCACGAGGCCCTGCACAACCACTACACCCAG
AAGTCCCTGTCCCTGTCCCCCGGCAAGGCCGCCGCCAACTCCTCCATCGACCTGATCTCCGTGCCCGTGGAC
TCCAGGAGGCCCGCCTGCAAGATCCCCAACGACCTGAAGCAGAAGGTGATGAACCACGACTACAAGGACGAC
GACGACAAGAGATGCGCCCCATCTATCAACAAGGACAACCTGAACCACGAGGACGGCGATGATGGCAACATC
AACAACAACGGAGACAATAATATTAATGGAGACGACAACAACATCAACAACAACATCAACGACAACAACAAC
TTCCTGCAGCAGCAACGCTACGACGAGGCCCTGTCCATCGAGGGCCTGTCTTGGGACGATATCACCGAGGAA
GAGCGGGACATCCTGATGTCCCTGCTGCTGAACAGATACATCAACGCCTCCATGCTGCCTTGGAACAATAAC
GGCATCCCCGTCGTGGTGAACGTGATCAGAAGCGCTCTGCCTCACAACCGGGGCCAGTTTATCGGCTACACC
GGCCTCCTGGAACTG
SEQ ID NO: 18 is a Wb5_P1-P2 peptide amino acid sequence:
QFPFFISPLLFFIIGTLAL
SEQ ID NO: 19 is a Wb5_P15-P17 peptide amino acid sequence:
INGDDNNINNNINDN
SEQ ID NO: 20 is a Wb5_P20-25 peptide amino acid sequence:
QQRYDEALSIEGLSWDDITEEERDILMSLLLNRYI
SEQ ID NO: 21 is a Wb5_P30-31 peptide amino acid sequence:
PWNNNGIPVVVNVIRSALP
SEQ ID NO: 22 is an exemplary tobacco etch virus protease cleavage site amino acid
sequence:
ENLYFQG
SEQ ID NO: 23 is an exemplary FLAG-tag amino acid sequence:
DYKDDDDK

### DETAILED DESCRIPTION

### I. Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “a protein” includes singular or plural proteins and can be considered equivalent to the phrase “at least one protein.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various aspects, the following explanations of terms are provided:

Antigen: A composition, such as a protein or peptide, that can stimulate the production of an immune response in a subject. An antigen reacts with the products of specific humoral or cellular immunity. In some examples, an antigen is a W. bancrofti antigen, such as Wb5.

Brugia malayi: A mosquito-borne roundworm that is a causative agent of lymphatic filariasis. The main vectors for B. malayi are Aedes and Mansonia mosquito species. Adult parasites reside in the lymphatics of the human host and are similar to those of W. bancrofti, but are smaller. Microfilariae (mf) are present in the circulation, primarily in peripheral blood. Mosquito hosts ingest microfilariae during a blood meal and they mature into L3 larvae. The larvae can then infect another human host during another blood meal. B. timori is similar to B. malayi, but with different geographical distribution, primarily limited to areas of Indonesia.

Contact: Placement in direct physical association; includes both in solid and liquid form. For example, contacting can occur in vitro with a protein and a sample in solution or on a substrate.

Detectable label: A compound or composition that is conjugated (e.g., covalently linked) directly or indirectly to another molecule (such as an antibody, for example, a secondary antibody) to facilitate detection of that molecule. Specific non-limiting examples of labels include fluorescent and fluorogenic moieties (e.g., fluorophores), chromogenic moieties, haptens (such as biotin, digoxigenin, and fluorescein), enzymes (such as horseradish peroxidase or alkaline phosphatase), affinity tags, and radioactive isotopes (such as ³²P, ³³P, ³⁵S, and ¹²⁵I). The label can be directly detectable (e.g., optically detectable) or indirectly detectable (for example, via interaction with one or more additional molecules that are in turn detectable). In some aspects herein, the detectable label includes an enzyme, such as horseradish peroxidase or alkaline phosphatase.

Epitope: The portion of an antigen that is recognized by an antibody or antigen receptor. Epitopes are also known as antigenic determinants. In some examples, the epitope is a W. bancrofti or Brugia sp. (such as B. malayi or B. timori) epitope, such as a Wb5 protein or a portion thereof.

Fusion protein: A protein containing amino acid sequence from at least two different (heterologous) proteins or peptides or a protein linked to a heterologous moiety (such as a non-peptide tag). In some examples herein, the fusion protein includes a Wb5 protein or portion thereof and one or more heterologous proteins or peptides. In some examples, the heterologous protein is a reporter protein (such as a luciferase protein). In other examples, the heterologous protein or moiety is a tag (such as a purification tag, for example a 6× histidine tag, glutathione-S-transferase (GST), an IgG Fc tag, or biotin).

Fusion proteins can be generated, for example, by expression of a nucleic acid sequence engineered from nucleic acid sequences encoding at least a portion of two different (heterologous) proteins. To create a fusion protein, the nucleic acid sequences must be in the same reading frame and contain no internal stop codons. Fusion proteins, particularly short fusion proteins, can also be generated by chemical synthesis.

Heterologous: A heterologous protein, polypeptide or nucleic acid refers to a protein, polypeptide or nucleic acid derived from a different source or species. A heterologous protein or polypeptide may also refer to a protein or polypeptide with an amino acid sequence that differs from a naturally occurring protein or polypeptide. Similarly, a heterologous nucleic acid refers to a nucleic acid with a nucleotide sequence that differs from a naturally occurring nucleic acid molecule.

Isolated: An “isolated” biological component (such as a nucleic acid molecule, protein, or cell) has been substantially separated or purified away from other biological components, such as chromosomal and extra-chromosomal DNA and RNA, proteins and cells. Nucleic acid molecules and proteins that have been “isolated” include those purified by standard purification methods. The term also embraces nucleic acid molecules and proteins prepared by recombinant expression in a host cell as well as chemically synthesized nucleic acid molecules and proteins. Isolated does not require absolute purity, and can include protein, peptide, or nucleic acid molecules that are at least 50% isolated, such as at least 75%, 80%, 90%, 95%, 98%, 99%, or even 99.9% isolated.

Lymphatic filariasis (LF): Lymphatic filariasis is caused by infection with filarial worms. W. bancrofti is responsible for about 90% of cases. The remaining cases are primarily caused by Brugia malayi, with a small number of cases caused by B. timori. These parasites are transmitted by mosquitoes. Most LF infections are asymptomatic, though infection can still damage the lymphatic system and kidneys. In some cases, LF develops into a chronic condition including lymphedema, elephantiasis, and hydrocele. An acute episode includes local inflammation of the skin, lymph nodes, and lymphatic vessels, and may accompany chronic lymphedema or elephantiasis.

Polypeptide, peptide or protein: A polymer in which the monomers are amino acid residues which are joined together through amide bonds. When the amino acids are alpha-amino acids, either the L-optical isomer or the D-optical isomer can be used. The terms “polypeptide,” “peptide,” and “protein” are used interchangeably herein. These terms apply to amino acid polymers in which one or more amino acid residue is an artificial chemical mimetic of a corresponding naturally occurring amino acid, as well as to naturally occurring amino acid polymers and non-naturally occurring amino acid polymers. The term “residue” or “amino acid residue” includes reference to an amino acid that is incorporated into a protein, polypeptide, or peptide.

A conservative substitution in a polypeptide is a substitution of one amino acid residue in a protein sequence for a different amino acid residue having similar biochemical properties. Typically, conservative substitutions have little to no impact on the activity of a resulting polypeptide. For example, a protein or peptide including one or more conservative substitutions (for example no more than 1, 2, 3, 4, 5, 6, 7, 8, 9, or 10 substitutions) retains the structure and function of the corresponding protein or peptide without the conservative substitution. A polypeptide can be produced to contain one or more conservative substitutions by manipulating the nucleotide sequence that encodes that polypeptide using, for example, standard procedures such as site-directed mutagenesis or PCR. In one example, such variants can be readily selected by testing protein activity or binding affinity (such as affinity for an antibody to the protein).

Examples of conservative substitutions are shown below.

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain.

The substitutions which in general are expected to produce the greatest changes in protein properties will be non-conservative, for instance changes in which (a) a hydrophilic residue, for example, seryl or threonyl, is substituted for (or by) a hydrophobic residue, for example, leucyl, isoleucyl, phenylalanyl, valyl or alanyl; (b) a cysteine or proline is substituted for (or by) any other residue; (c) a residue having an electropositive side chain, for example, lysyl, arginyl, or histadyl, is substituted for (or by) an electronegative residue, for example, glutamyl or aspartyl; or (d) a residue having a bulky side chain, for example, phenylalanine, is substituted for (or by) one not having a side chain, for example, glycine.

Recombinant: A recombinant nucleic acid molecule or protein is one that has a sequence that is not naturally occurring or has a sequence that is made by an artificial combination of two otherwise separated segments of sequence. This artificial combination can be accomplished by chemical synthesis or by the artificial manipulation of isolated segments of nucleic acid molecules, such as by genetic engineering techniques. The term “recombinant” also includes nucleic acids and proteins that have been altered solely by addition, substitution, or deletion of a portion of the natural nucleic acid molecule or protein.

Sample: Refers to any biological sample that includes or may include an analyte of interest, such as antibodies to W. bancrofti or B. malayi. In some aspects, the sample is a biological sample obtained from a subject, such as a blood, serum, or plasma sample.

Sensitivity and specificity: Statistical measurements of the performance of a binary classification test. Sensitivity measures the proportion of actual positives which are correctly identified (e.g., the percentage of samples that are identified as including antibodies from a particular organism). Specificity measures the proportion of negatives which are correctly identified (e.g., the percentage of samples that are identified as not including antibodies from a particular organism).

Subject: Living multi-cellular vertebrate organisms, a category that includes human and non-human mammals. In some aspects herein, the subject is a human, veterinary, or laboratory subject.

Substrate: A solid support or surface. The configuration of the solid support can be flat (e.g., a plate or slide), spherical (e.g., a bead), or another configuration. Suitable substrate materials include, but are not limited to organic polymers such as nitrocellulose, polypropylene, polyethylene, polybutylene, polyisobutylene, polybutadiene, polyisoprene, polyvinylpyrrolidine, polytetrafluroethylene, polyvinylidene difluroide, polyfluoroethylene-propylene, polyethylenevinyl alcohol, polymethylpentene, polycholorotrifluoroethylene, polysulfornes, hydroxylated biaxially oriented polypropylene, aminated biaxially oriented polypropylene, thiolated biaxially oriented polypropylene, ethyleneacrylic acid, thylene methacrylic acid, and blends of copolymers thereof. In general, the material used for the substrate is amenable to surface activation such that upon activation, the surface of the substrate is capable of covalently attaching a biomolecule, such as a Wb5 protein or portion thereof.

Vector: A vector is a nucleic acid molecule allowing insertion of foreign nucleic acid without disrupting the ability of the vector to replicate and/or integrate in a host cell. A vector can include nucleic acid sequences that permit it to replicate in a host cell, such as an origin of replication. A vector can also include one or more selectable marker genes and other genetic elements. An expression vector is a vector that contains the necessary regulatory sequences to allow transcription and translation of inserted gene or genes.

Wuchereria bancrofti: A mosquito-borne roundworm that is the major causative agent of lymphatic filariasis. The lifecycle of W. bancrofti is carried out in humans and mosquitoes. Adult parasites reside in the lymphatics of the human host and first stage larvae (“microfilariae;” mf) are present in the circulation, primarily in peripheral blood. The microfilariae migrate between the deep and peripheral circulation with a diumal periodicity, being present in the deep veins during the day and the peripheral circulation during the night. Mosquito hosts (such as Culex, Anopheles, or Aedes species) ingest microfilariae during a blood meal and they mature into L3 larvae. The larvae are then deposited from the mosquito mouthparts onto the skin of a human host during another blood meal. The larvae reside in the lymph nodes, primarily in the leg and genital areas, and develop into adult worms in about one year. The adults mate and produce microfilariae, and the lifecycle is repeated.

### II. Wb5 Proteins, Nucleic Acids, and Vectors

Disclosed herein are W. bancrofti or B. malayi proteins that can be used for specific and sensitive detection of one or more agents causing lymphatic filariasis, such as W. bancrofti or B. malayi. In aspects, the protein is a Wb5 protein or a portion thereof (such as an immunoreactive antigen or epitope of Wb5). In some aspects, the Wb5 protein or portion thereof is specifically bound by an antibody in a sample from a subject infected with W. bancrofti. The antibody may be any immunoglobulin type. In some examples, the antibody is an IgG immunoglobulin type, such as IgG4. In additional aspects, one or more disclosed Wb5 protein, portion thereof, or a fusion protein including a Wb5 protein or portion thereof covalently linked to a substrate is provided.

In some aspects, the Wb5 protein has at least 90% sequence identity (such as at least 90%, at least 91%, at least 92%, at least 93%, at least 94%, at least 95%, at least 96%, at least 97%, at least 98%, at least 99%, or more sequence identity) to the amino acid sequence of any one of SEQ ID NO: 2, SEQ ID NO: 4, SEQ ID NO: 8, or SEQ ID NO: 10. In some examples, the Wb5 protein includes or consists of the amino acid sequence of any one of SEQ ID NO: 2, SEQ ID NO: 4, SEQ ID NO: 8, or SEQ ID NO: 10. In other aspects, a portion of Wb5 protein, such as a portion of the Wb5 protein that retains ability to specifically bind to antibodies present in a subject infected with W. bancrofti, are contemplated. The functional portion may include at least about 10% (such as at least about 10%, 15%, 20%, 25%, 30%, 40%, 50%, 60%, 70%, 80%, 90%, 95%, or more) of the amino acid sequence of any one of SEQ ID NO: 2, SEQ ID NO: 4, SEQ ID NO: 8, or SEQ ID NO: 10.

In some examples, the portion of the Wb5 protein includes an immunoreactive portion or one or more epitopes, for example, is recognized by an antibody or antigen receptor. In some examples, the portion of the Wb5 protein is about 10-35 amino acids in length, for example about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, or about 35 amino acids long. In one example, the portion of the Wb5 protein is 15 amino acids long. In another example, the Wb5 protein is 19 amino acids long. In an additional example, the Wb5 protein is 35 amino acids long. In specific examples, the portion of the Wb5 protein has at least 90% sequence identity (such as at least 90%, at least 91%, at least 92%, at least 93%, at least 94%, at least 95%, at least 96%, at least 97%, at least 98%, at least 99%, or more sequence identity) to any one of SEQ ID NOs: 18-21 or includes or consists of the amino acid sequence of any one of SEQ ID NOs: 18-21.

In some examples, the Wb5 polypeptide includes a signal peptide (e.g., Wb5A, SEQ ID NO: 4). In other examples, the Wb5 polypeptide does not include the signal peptide sequence (e.g., Wb5B, SEQ ID NO: 2). Thus, in some examples, the disclosed Wb5 polypeptides do not include a starting methionine (e.g., SEQ ID NO: 2). In other aspects, the Wb5 polypeptide is expressed with one or more tags (such as a purification tag), which may optionally be cleaved prior to use. In some examples, the polypeptide includes a tobacco etch virus (TEV) protease cleavage site (e.g., ENLYFQG; SEQ ID NO: 22). In some examples, the one or more tags are N-terminal to the Wb5 polypeptide, C-terminal to the Wb5 polypeptide, or both. Exemplary tags include a polyhistidine tag (such as a 6×His tag), glutathione-S-transferase (GST), an IgG-Fc tag, a FLAG-tag (e.g., DYKDDDDK; SEQ ID NO: 23), or biotin.

Also provided are fusion proteins including a disclosed Wb5 protein or portion thereof. In some examples, the fusion protein includes a Wb5 protein (e.g., SEQ ID NO: 2, SEQ ID NO: 4, SEQ ID NO: 8, or SEQ ID NO: 10) or portion thereof that is linked to a reporter protein or tag. In other examples, the fusion protein includes a portion of a Wb5 protein (e.g., SEQ ID NOs: 18-21) that is linked to a reporter protein or tag. The reporter protein or tag may be N-terminal to the Wb5 protein or portion thereof, C-terminal to the Wb5 protein or portion thereof, or both. In some examples, the fusion protein has at least 90% sequence identity (such as at least 90%, at least 91%, at least 92%, at least 93%, at least 94%, at least 95%, at least 96%, at least 97%, at least 98%, at least 99%, or more sequence identity) to the amino acid sequence of SEQ ID NO: 14 or SEQ ID NO: 16. In some examples, the fusion protein includes or consists of the amino acid sequence of SEQ ID NO: 14 or SEQ ID NO: 16.

The reporter protein may be any protein that is capable of generating a detectable signal. In some examples, the reporter protein is an enzyme, such as luciferase, horseradish peroxidase, or alkaline phosphatase. In other examples, the reporter protein is a fluorescent protein, such as a green fluorescent protein or red fluorescent protein. In a specific example, the reporter protein is Renilla luciferase. In other examples, the reporter protein does not directly generate a detectable signal, but is a protein for which antibodies are available, and which can be detected. In some examples, the protein is GST or maltose-binding protein (MBP).

Further provided are nucleic acid molecules (e.g., DNA, cDNA, RNA or mRNA) encoding the Wb5 polypeptides and fusion proteins disclosed herein. Unless otherwise specified, a “nucleic acid molecule encoding a polypeptide” includes all nucleotide sequences that are degenerate versions of each other and encode the same amino acid sequence. For example, a polynucleotide encoding a Wb5 polypeptide includes a nucleic acid sequence that is degenerate as a result of the genetic code. There are 20 natural amino acids, most of which are specified by more than one codon. Therefore, all degenerate nucleotide sequences are included as long as the amino acid sequence of the polypeptide encoded by the nucleotide sequence is unchanged.

In some aspects, the disclosed polypeptide sequences are back-translated to codon optimized DNA. In some examples, the nucleic acid encoding the Wb5 protein is codon-optimized for the cell in which it is to be expressed (such as a bacterial cell, an insect cell, or a mammalian cell). Codon usage bias, the use of synonymous codons at unequal frequencies, is ubiquitous among genetic systems. The strength and direction of codon usage bias is typically related to genomic G+C content and the relative abundance of different isoaccepting tRNAs. Codon usage can affect the efficiency of gene expression. Codon-optimization refers to replacement of at least one codon (such as at least 5 codons, at least 10 codons, at least 25 codons, at least 50 codons, at least 75 codons, at least 100 codons or more) in a nucleic acid sequence with a synonymous codon (one that codes for the same amino acid) more frequently used (preferred) in the organism in which the nucleic acid is to be expressed. Each organism has a particular codon usage bias for each amino acid, which can be determined from publicly available codon usage tables (for example see Nakamura et al., Nucleic Acids Res. 28:292, 2000 and references cited therein), databases (e.g., kazusa.or.jp/codon), or commercial sources. One of skill in the art can modify a nucleic acid encoding a particular amino acid sequence, such that it encodes the same amino acid sequence, while being optimized for expression in a particular cell type or organism. In particular examples, the Wb5 sequence is codon-optimized for expression in mammalian cells (such as human cells).

In some aspects, the Wb5 protein is encoded by a nucleic acid having at least 90% sequence identity (such as at least 90%, at least 91%, at least 92%, at least 93%, at least 94%, at least 95%, at least 96%, at least 97%, at least 98%, at least 99%, or more sequence identity) to the nucleic acid sequence of SEQ ID NO: 3. In some examples, the Wb5 protein is encoded by a nucleic acid that includes or consists of the amino acid sequence of SEQ ID NO: 3. In other aspects, the Wb5 protein is encoded by a nucleic acid having at least 90% sequence identity (such as at least 90%, at least 91%, at least 92%, at least 93%, at least 94%, at least 95%, at least 96%, at least 97%, at least 98%, at least 99%, or more sequence identity) to the nucleic acid sequence of SEQ ID NO: 1. In some examples, the Wb5 protein is encoded by a nucleic acid that includes or consists of the amino acid sequence of SEQ ID NO: 1. In additional aspects, the Wb5 protein is encoded by a nucleic acid having at least 90% sequence identity (such as at least 90%, at least 91%, at least 92%, at least 93%, at least 94%, at least 95%, at least 96%, at least 97%, at least 98%, at least 99%, or more sequence identity) to the nucleic acid sequence of any one of SEQ ID NOs: 11-13 or includes or consists of the nucleic acid sequence of any one of SEQ ID NOs: 11-13.

In other aspects, the Wb5 protein is encoded by a native nucleic acid sequence. Exemplary Wb5 nucleic acid sequences are available in public databases, for example WormBase ParaSite Accession No. PRJNA275548-pt002 (pairedcontig_1997:12795-13055) or PRJEB536 (WBA_contig0003917:508-768), which are incorporated herein by reference as present in the database on May 31, 2022. In some examples, the Wb5 protein is encoded by a nucleic acid with at least 95% sequence identity to the nucleic acid sequence of any one of SEQ ID NOs: 5-7 or 9, or a nucleic acid including or consisting of any one of SEQ ID NOs: 5-7 or 9, or a portion thereof.

In additional aspects, the fusion protein is encoded by a nucleic acid having at least 90% sequence identity (such as at least 90%, at least 91%, at least 92%, at least 93%, at least 94%, at least 95%, at least 96%, at least 97%, at least 98%, at least 99%, or more sequence identity) to the nucleic acid sequence of SEQ ID NO: 15 or SEQ ID NO: 17 or includes or consists of the nucleic acid sequence of SEQ ID NO: 15 or SEQ ID NO: 17.

Minor alterations of nucleic acids encoding a Wb5 polypeptide primary amino acid sequence are also contemplated herein. Such alterations to the nucleic acid may result in polypeptides that have substantially equivalent activity as compared to the starting counterpart polypeptide described herein. Such alterations may be deliberate, for example as by site-directed mutagenesis, or may be spontaneous. All of the nucleic acids produced by these alterations are included herein. Thus, a specific, non-limiting example of an altered nucleic acid encoding a disclosed polypeptide is a nucleic acid encoding conservative variant of the polypeptide (such as encoding a single conservative amino acid substitution, for example, one or more conservative amino acid substitutions, for example 1-10 conservative substitutions, 2-5 conservative substitutions, 4-9 conservative substitutions, such as 1, 2, 5 or 10 conservative substitutions). In other examples, the nucleic acid may encode a polypeptide encoding one or more non-conservative substitutions (for example, encoding 1-10 non-conservative substitutions, 2-5 non-conservative substitutions, 4-9 non-conservative substitutions, such as 1, 2, 5 or 10 non-conservative substitutions), so long as the encoded polypeptide retains at least one Wb5 structural or functional property, such as specific binding to anti-Wb5 antibodies, for example, from a subject infected with W. bancrofti.

Vectors that include the disclosed nucleic acid molecules are also provided. DNA sequences encoding the disclosed polypeptides can be expressed in vitro or in vivo by DNA transfer into a suitable host cell. The cell may be prokaryotic or eukaryotic. Methods of stable transfer, meaning that the foreign DNA is continuously maintained in the host, are known in the art. Polynucleotide sequences encoding the disclosed polypeptides can be operably linked to expression control sequences, such as heterologous expression control sequences (such as a heterologous promoter). An expression control sequence operably linked to a coding sequence is joined such that expression of the coding sequence is achieved under conditions compatible with the expression control sequences. The expression control sequences include, but are not limited to, appropriate promoters, enhancers, transcription terminators, a start codon in front of a protein-encoding gene, splicing signal for introns, maintenance of the correct reading frame of that gene to permit proper translation of mRNA, and stop codons. In one example, a pET30A vector can be used for expression in E. coli. In another example, a pFastBac (such as pFastBacgp67) baculovirus expression vector can be used for expression in Sf9 cells. In a further example, a pcDNA3 (such as pcDNA3.4) mammalian expression vector can be used for expression in mammalian cells (such as HEK293 cells).

Host cells can include microbial, yeast, insect, and mammalian organisms. Methods of expressing DNA sequences having eukaryotic or viral sequences in prokaryotes are well known in the art. Non-limiting examples of suitable host cells include bacteria, archaea, insect (for example, Spodoptera frugiperda cells), fungi (for example, yeast), plant, and animal cells (for example, mammalian cells, such as human cells). Exemplary cells of use include Escherichia coli, Spodoptera frugiperda Sf9 cells, and immortalized mammalian cell lines. Examples of commonly used mammalian host cell lines are VERO cells, HeLa cells, CHO cells, HEK293 cells (e.g., 293-F cells), W138 cells, BHK cells (such as BHK21 cells), HT-1080 cells, PER.C6 cells, HKB-11 cells, HuH-7 cells, and COS cells, although other cell lines may be used, such as cells designed to provide higher expression, desirable glycosylation patterns, or other features.

Transformation of a host cell with recombinant DNA can be carried out by techniques known to those skilled in the art. Where the host is prokaryotic, such as, but not limited to, E. coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired, or by electroporation. When the host is a eukaryote, methods of transfection of DNA such as calcium phosphate coprecipitates, mechanical procedures such as microinjection, electroporation, insertion of a plasmid encased in liposomes, or virus vectors can be used.

### III. Methods of Detection and Diagnosis

Provided herein are methods of detecting lymphatic filariasis (such as W. bancrofti or Brugia sp. infection) in a subject, for example by detecting presence of antibodies to a Wb5 protein or portion thereof in a sample from a subject. In some examples, the methods include detecting presence of microfilarial stage infection in the subject. In some aspects, the methods utilize a luciferase immunoprecipitation system (LIPS) assay. In other aspects, the methods utilize an immunoassay method, such as an ELISA, lateral flow assay, or a bead-based assay (such as a Luminex® assay).

In some aspects, the disclosed methods are specific for detection of W. bancrofti and/or Brugia sp. infection. In some examples, the disclosed methods specifically detect antibodies to W. bancrofti (such as antibodies that specifically bind to Wb5 protein or a portion thereof). In other examples, the methods include detection of antibodies to B. malayi or B. timori (such as antibodies that specifically bind to Wb5 protein or a portion thereof). In some examples, the Wb5 protein does not cross-react with antibodies produced by infection of a subject with Loa loa, Onchocerca volvulous, or Strongyloides. In some aspects, the disclosed methods have a specificity of 90% or more (such as at least 90%, at least 91%, at least 92%, at least 93%, at least 94%, at least 95%, at least 96%, at least 97%, at least 98%, at least 99%, at least 99.5%, at least 99.9%, or even 100%). In one example, the disclosed methods have a specificity of 100%. In additional aspects, the disclosed methods have a sensitivity of 60% or more (such as at least 60%, at least 65%, at least 70%, 75%, at least 80%, at least 85%, at least 90%, at least 95%, or more). In one example, the disclosed methods have a sensitivity of at least 93%. In a particular example, the disclosed methods have a specificity of 100% and a sensitivity of 93.75%.

Appropriate samples include any biological sample, including samples obtained from a human or animal subject. Suitable samples include all biological samples useful for detection of W. bancrofti or Brugia sp. infection in subjects, including, but not limited to, bodily fluids (for example, blood, serum, plasma, lymph, or saliva). In some examples, the subject is a human subject. In some examples, the sample is used directly in the methods described herein, or with minimal processing, such as cell lysis or addition of water or buffer.

In some aspects, the sample is from a subject or a group of subjects that are infected with or suspected to be infected with W. bancrofti. In other examples, the sample is from a subject or a group of subjects that are infected with or suspected to be infected with B. malayi or B. timori. In some examples, the sample is from a subject or group of subjects that live in an area where W. bancrofti, B. malayi, and/or B. timori are endemic. W. bancrofti and B. malayi are endemic in tropic and sub-tropic regions of Southeast Asia, Africa, the Indian subcontinent, the Pacific islands, and portions of the Caribbean, Latin America, and South America. B. timori is endemic to regions of Indonesia.

In some aspects, a sample is from a single subject. In other aspects, the sample is a pooled sample obtained by mixing samples from a plurality of subjects. In some examples, a pooled sample includes samples from 2 or more subjects (for example, 2, 3, 4, 5, 6, 7, 8, 9, 10, 15, 20, or more subjects). In other examples, a pooled sample includes samples from 2-5 subjects, 3-8 subjects, 5-10 subjects, 8-15 subjects, or 12-20 subjects. In some examples, the pooled sample is from a population of subjects in a specified geographical region (such as a village, town, city, or endemic region). In other examples, the pooled sample is from a population of subjects with the same infection status across more than one geographical region.

In some aspects of the disclosed methods, the methods include using a luciferase immunoprecipitation system (LIPS) assay. In some examples, the method includes contacting a fusion protein comprising a Wb5 protein or portion thereof and a reporter protein with a sample from a subject to form a complex between the Wb5 protein or a portion thereof and an antibody to W. bancrofti (or Brugia sp.) present in the sample. The complex is contacted with an immobilized binding agent capable of binding to the antibody to W. bancrofti (or Brugia sp.) to form an immobilized complex including the fusion protein. Output from the reporter protein in the immobilized complex is detected, thereby detecting presence of antibodies to W. bancrofti or Brugia sp. in the sample. The method can be carried out in any format, such as in a multiwell plate.

The order of addition of the reagents and sample is not critical. Thus, the sample may be mixed with the reporter protein and then the immobilized binding agent could be added. Alternatively, the sample may be mixed with the immobilized binding agent, and then the reporter protein may be added to the mixture. Finally, the sample, reporter protein, and immobilized binding agent could be mixed together at the same time, or the reporter protein and immobilized binding agent may be pre-mixed and the sample subsequently added to the mixture.

In particular examples, the reporter protein is a luciferase protein, such as Renilla luciferase. In such an example, detecting output from the reporter protein includes adding a luciferase substrate, for example coelenterazine. Other reporter proteins can be used, and one of skill in the art can select an appropriate substrate (if the reporter protein is an enzyme) or other mode of detection, such as fluorescence detection (if the reporter protein is a fluorescent protein such as GFP).

In some examples, the immobilized binding agent is protein A, protein G, or a combination thereof, such as a fusion protein including Fc binding domains from both protein A and protein G (e.g., protein A/G). In one example, the immobilized binding agent is protein A/G beads, such as protein A/G Sepharose or protein A/G magnetic beads.

In additional aspects, the disclosed methods include using an ELISA assay, such as an “indirect” ELISA assay to detect Wb5 antibodies in a sample from a subject. Enzyme-linked immunosorbent assay (ELISA) is a test that uses antibodies and color change to identify a substance. In one type of ELISA, an antigen (such as a Wb5 protein or portion thereof) is attached to a surface, and the remainder of the surface is then usually blocked to prevent non-specific binding of later-added components of the assay. A sample (such as a sample from a subject that is infected with or suspected to be infected with W. bancrofti or Brugia sp.) is applied over the surface so it can bind to the antigen. A secondary antibody (such as an anti-human antibody) is then applied. The secondary antibody is linked to a detectable label, such as an enzyme, and presence of Wb5 antibodies in the sample is detected. In some examples, a substance containing the enzyme's substrate is added. The subsequent reaction produces a detectable signal, most commonly a color change in the substrate. Other types of ELISAs include sandwich ELISA, competitive ELISA, and multiple and portable ELISA.

In some examples, the Wb5 protein or portion thereof is covalently linked to a surface, such as a well of a multiwell plate. Between each step, the plate is typically washed with a mild detergent solution to remove any proteins or antibodies that are not specifically bound. After the final wash step, the plate is developed by adding an enzymatic substrate to produce a visible signal (or otherwise visualized, depending on the exact detection system being used), which indicates the presence and/or amount of Wb5 antibodies in the sample.

In some aspects of the disclosed methods, detecting presence of antibodies to Wb5 includes using a lateral flow assay (LFA) to detect presence of antibodies to Wb5 in a sample. LFA is a simple, rapid, portable and low-cost method for detecting an analyte in a sample. In view of these features, LFA is well-suited for point-of-care diagnostics, particularly in settings where rapid test results are necessary.

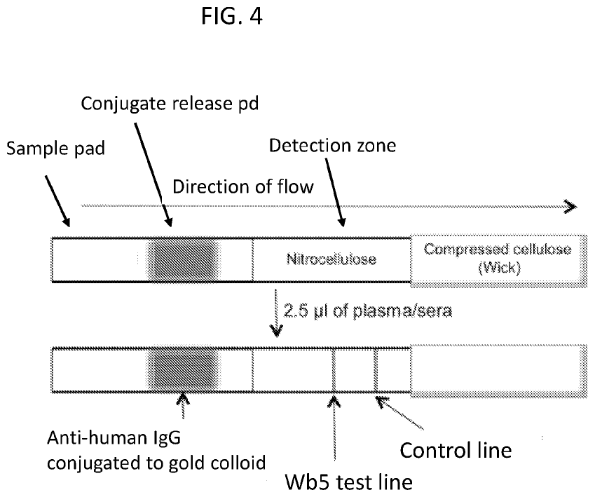

LFA uses a fluid sample that contains, or is suspected of containing, the analyte of interest (such as antibodies to Wb5), which moves via capillary action through various zones of a paper test strip. The lateral flow test strip contains multiple zones of polymeric strips on which molecules capable of interacting with the analyte of interest are attached. Typically, a lateral flow test strip is made up of overlapping membranes that are mounted on a stable backing card. An exemplary test strip is shown in FIG. 4. To perform the assay, the sample (such as blood, plasma, or serum) is applied to an adsorbent sample pad located at one end of the lateral flow test strip. The sample pad is impregnated with buffer salts and surfactants that make the sample suitable for interaction with the detection system. The sample pad also holds any excess of the fluid sample and once soaked, the fluid flows to the conjugate release pad, which contains labeled antibodies specific to the target analyte. The labeled antibodies are typically conjugated to colored or fluorescent particles, such as colloidal gold or latex microspheres. In some aspects herein, the conjugate release pad contains anti-human IgG conjugated to gold colloid. If the target analyte is present in the fluid sample, the labeled antibodies bind the analyte and the fluid containing the conjugates continue their migration to the detection zone, which contains a test line and a control line. The detection zone is generally composed of nitrocellulose and contains specific biological components (such as antibodies or antigens) immobilized in lines. The test line will show a signal if the target analyte is present in the sample. In the context of the methods disclosed herein, the test line is Wb5 protein or a portion thereof. The control line typically contains affinity ligands that provide an indication of whether the sample has properly migrated along the paper strip and that the reagents in the conjugate pad are active. Therefore, a signal on the control line indicates that the fluid sample has properly migrated along the test strip as it will be positive regardless of whether the target analyte is present in the sample being tested. In some examples, the control line is human IgG or human IgG4. To maintain capillary flow along the test strip an absorbent pad is included at the end of the paper strip. The absorbent pad wicks away excess reagents and prevents backflow of the liquid. The results of a LFA can be read visually (by eye) or by using a lateral flow reader.

Exemplary LFAs are described in U.S. Pat. Nos. 6,136,610; 7,871,781; and 10,048,251; and U.S. Patent Application Publication Nos. 2003/0119203; 2007/0020699; 2010/00015658; 2013/0137189; and 2018/0149600, each of which is herein incorporated by reference in its entirety.

In some aspects, the disclosed methods further include detecting presence of antibodies to W. bancrofti Wbl23 protein in the sample. Methods of detecting antibodies to Wb123 are described in U.S. Pat. No. 9,068,993, incorporated herein by reference in its entirety. In some examples, the methods include detecting antibodies to Wb5 and Wb123 in a single assay (such as a multiplex assay). In other examples, antibodies to Wb5 and Wb123 are detected in separate (e.g., parallel or sequential) assays using portions of the same sample from a subject. In some aspects, the assay for detecting Wbl23 antibodies is the same type of assay as that for detecting Wb5 antibodies (such as a LIPS, ELISA, Luminex®, or LFA). In other aspects, the assay for Wbt23 antibodies is a different type of assay as that used for detecting Wb5 antibodies.

In some aspects, a subject is treated for lymphatic filariasis when Wb5 antibodies are detected in a sample from the subject. In some examples, the treatment includes diethylcarbamazine (DEC). In some examples, DEC is administered at 6 mg/kg/day for 1 day or 12 days. DEC is contraindicated in patients who may also have onchocerciasis. In such case, the subject may be administered doxycycline (such as 200 mg/day) for 4-6 weeks.

In other aspects, a lymphatic filariasis control regimen is selected for a population when antibodies to Wb5 protein are detected in a pooled sample from the population. In further aspects, the lymphatic filariasis control regimen is implemented in the population. In some examples, the control regimen is mass drug administration (MDA) of antifilarial medications to the population. In one example, the MDA is single dose treatment with a combination of ivermectin, DEC, and albendazole (such as 200 μg/kg ivermectin, 6 mg/kg DEC, and 400 mg albendazole). In other examples, the MDA is a two-drug regimen of DEC plus albendazole, ivermectin plus albendazole, or albendazole alone. The particular regimen depends on the presence of other co-endemic filarial diseases. For example, albendazole alone is used in areas where loiasis is co-endemic; ivermectin and albendazole is used in areas where onchocerciasis is present; and DEC and albendazole or ivermectin, DEC, and albendazole is used in areas without onchocerciasis.

### IV. Kits

Further provided herein are kits for detecting W. bancrofti or Brugia sp. antibodies (such as Wb5 antibodies) in a sample. In some aspects, the kit includes a fusion protein including a Wb5 protein or portion thereof linked to a reporter protein and an immobilized binding agent capable of binding to the antibody to W. bancrofti or Brugia sp. In some examples, the Wb5 protein or portion thereof is a protein with an amino acid sequence at least 95% identity to any one of SEQ ID NOs: 2, 4, 8, 10, or 18-21, or is a protein including or consisting of the amino acid sequence of any one of SEQ ID NOs: 2, 4, 8, 10, or 18-21. In some examples the immobilized binding agent includes protein A, protein G, or protein A/G, such as protein A/G Sepharose or protein A/G magnetic beads. In one example, the fusion protein is Wb5 protein or a portion thereof linked to luciferase. In some aspects, the kit further includes a substrate for the reporter protein, which in one example is the luciferase substrate coelenterazine. In other aspects, the kit includes a Wb5 protein or portion thereof linked to a tag (such as a His tag, GST, Ig Fc, FLAG-tag, or biotin).

In other aspects, the kit includes a Wb5 protein or portion thereof linked to a substrate. In some examples, the Wb5 protein or portion thereof is a protein with an amino acid sequence at least 95% identity to any one of SEQ ID NOs: 2, 4, 8, 10, or 18-21, or is a protein including or consisting of the amino acid sequence of any one of SEQ ID NOs: 2, 4, 8, 10, or 18-21. In some examples, the substrate is a lateral flow test strip. In other examples, the substrate is a multiwell plate. In other examples, the substrate is a bead, such as a magnetic bead. The kit may further include additional reagents for detection of Wb5 antibodies, such as one or more of a buffer, a detectably labeled secondary antibody, and substrate for the detectable label (for example, when the detectable label is an enzyme).

In additional aspects, the kit may also include reagents for detection of antibodies to Wb123 in a sample.

### EXAMPLES

The following examples are provided to illustrate particular features of certain aspects of the disclosure, but the scope of the claims should not be limited to those features exemplified.

### Example 1

### Screening Candidate Proteins

All-vs-All blast analyses were performed for all filarial encoded proteins. Proteins of W. bancrofti (from any of the genomes) that only exhibited homology with W. bancrofti or with B. malayi alone (for pan-LF) and no homology with O. volvulus, L. loa were shortlisted. Further, evidence of transcriptional and/or proteomic expression in mf or L3 stages of W. bancrofti and/or B. malayi was used to further down-select targets.

Twelve identified proteins (expressed in slightly different forms based on Wb-genome origins) were cloned into pREN2 constructs and expressed as fusion proteins in 293F cells. Fusion proteins were made by cloning the genes coding for the proteins into a FLAG epitope-tagged mammalian Renilla reniformis luciferase (Ruc)-containing expression vector pREN2. Lysates containing the fusion proteins were prepared by transfecting 293F cells (Thermo Fisher Scientific, Waltham, MA) as per the manufacturer's instructions. Briefly, 30 μg of plasmid was used to transfect 293F cells at a final concentration of 1 μg of plasmid for 1×10⁶ cells in FreeStyle 293 Expression Medium (Thermo Fisher Scientific) and cultured for 48 hours at 37° C., 8% CO₂, and shaking at 125 rpm. The cells were centrifuged, the pellet was lysed, and the lysate was frozen until used. A standard luciferase immunoprecipitation system (LIPS) antibody assay was used for the evaluation of IgG and IgG4 responses to each of the antigens. Briefly, patient sera were diluted 1:10 in assay buffer A (20 mM Tris, pH 7.5, 150 mM NaCl, 5 mM MgCl₂, 1% Triton X-100) in a 96-well polypropylene microtiter plate. For evaluating antibody titers by LIPS, 40 μl of buffer A, 10 μl of diluted human sera (1-μl equivalent), and 50 μl of 1×10⁶ luminescence units (LU) of Ruc antigen from the lysate, diluted in buffer A, were added to each well of a second polypropylene plate, which was used to conduct the assay. This plate, containing 100 μl of the antigen-antibody reaction mixture, was then incubated for 30 minutes at room temperature. Next, 7 μl of a 30% suspension of Protein A/G beads (Pierce, Rockford, IL) in phosphate-buffered saline was added to the bottom of a 96-well filter high-throughput-screening plate (Millipore, Bedford, MA). The 100-μl antigen-antibody reaction mixture from each microtiter well was then transferred to the well of the filter plate, and this plate was further incubated for 30 minutes at room temperature on a rotary shaker. The filter plate containing the mixture was then applied to a vacuum manifold. The retained protein A/G beads were washed, and after the final wash, the plate was blotted and LU measured with a Berthold LB 960 Centro microplate luminometer, using a coelenterazine substrate mixture (Promega, Madison, WI). All LU data presented were obtained from the averages for two independent experiments and corrected for background by subtracting LU values of beads incubated with respective extracts but no sera.

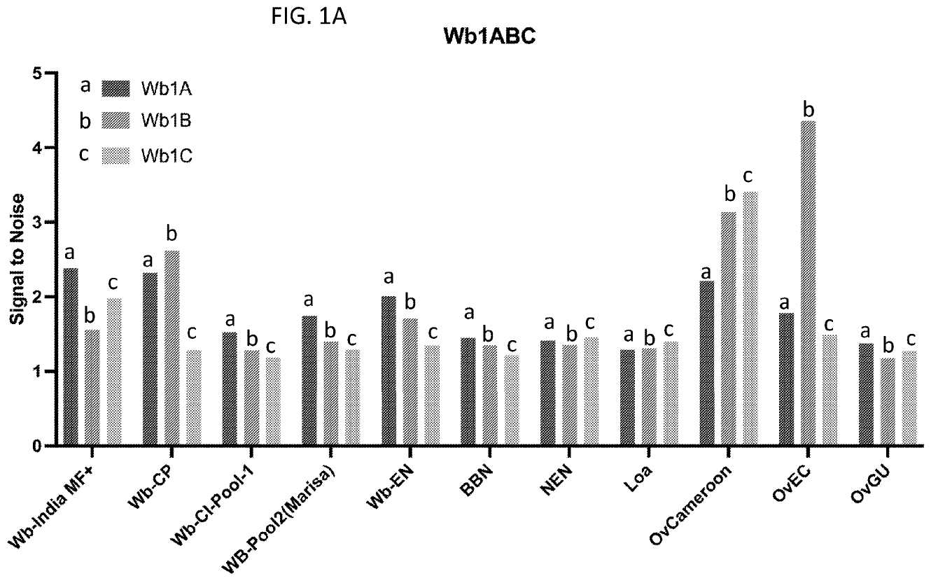

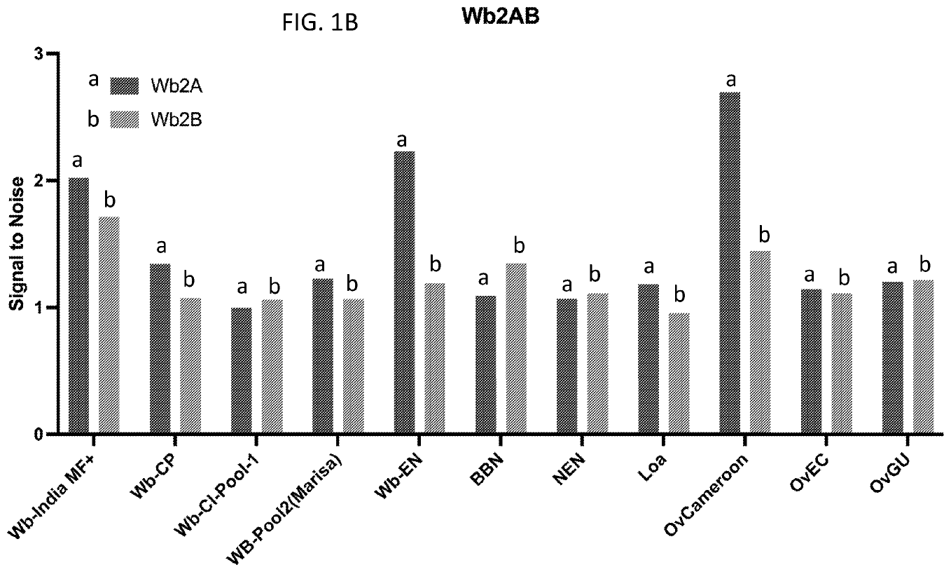

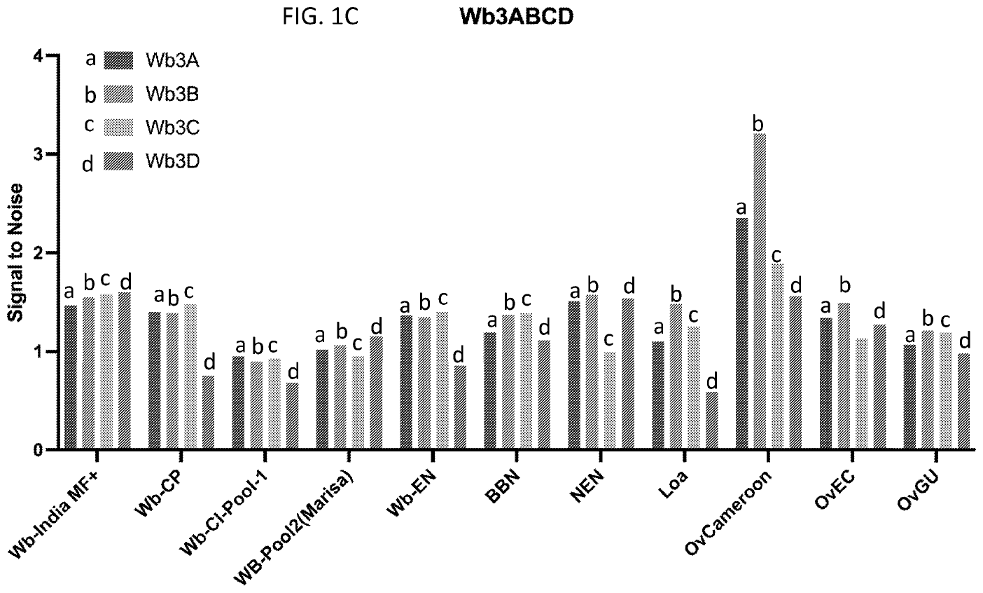

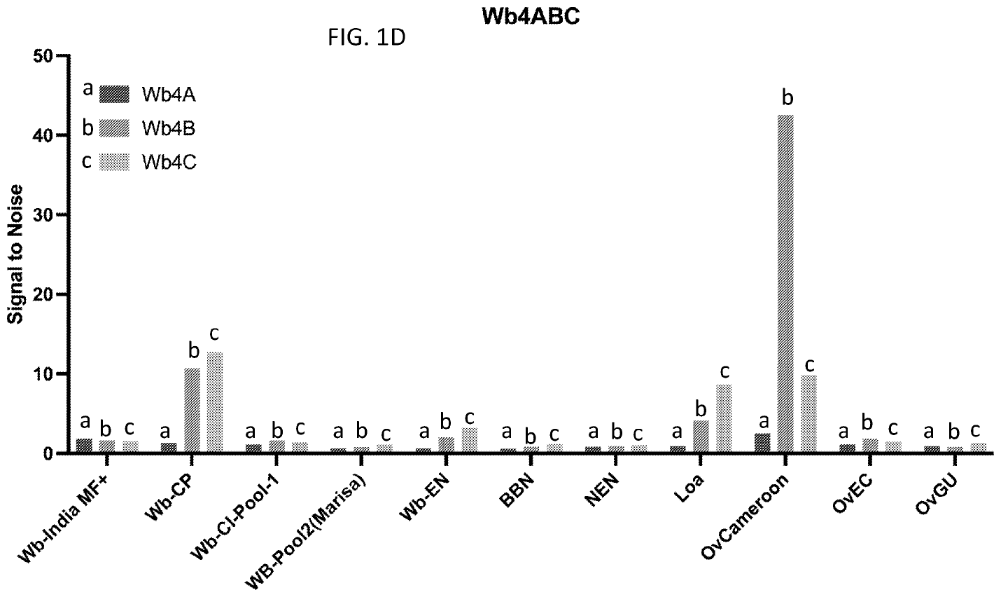

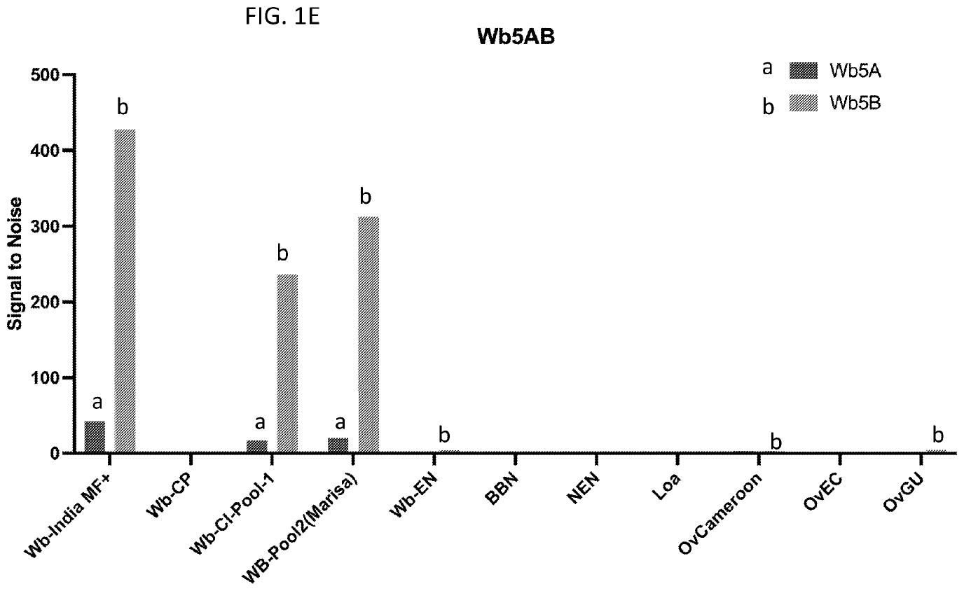

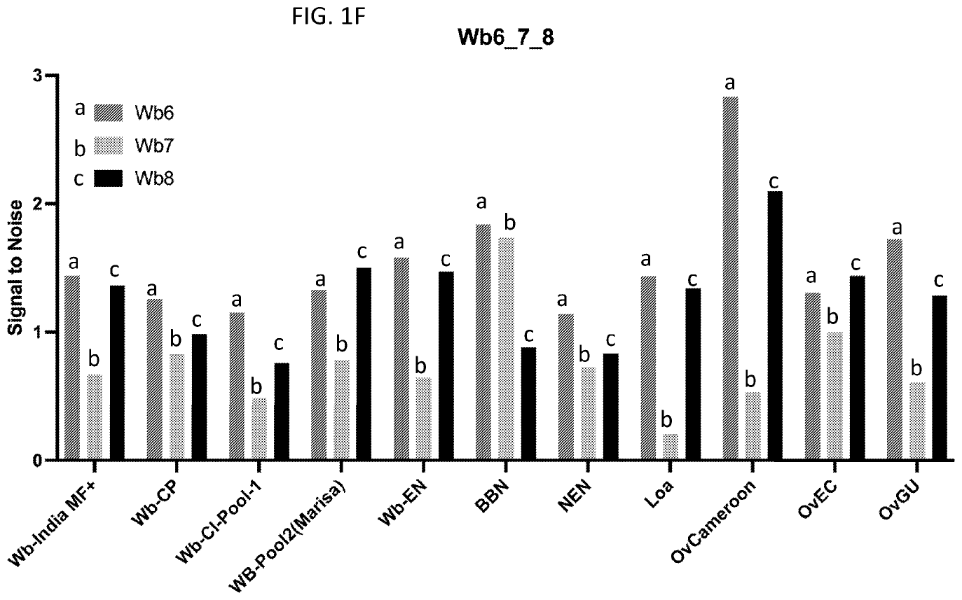

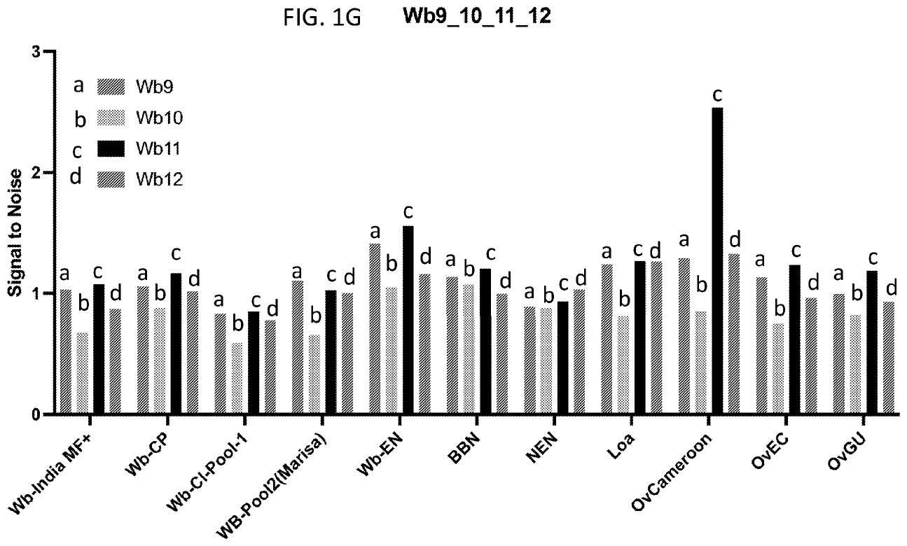

The lysates containing the fusion proteins were screened by LIPS assay individually with pooled sera from Wb-infected mt-positive sera (Wb-lndia_MF+; Wb-CI-Pool1; Wb-CI-Pool2), Wb-chronic pathology (lymphedema), endemic normals from India; healthy blood bank normals (BBN, NEN), Loa-infected; and Onchocerca-infected (Cameroon, Ecuador & Guatemala). FIGS. 1A-1G represent the signal to noise values of each of the proteins for total IgG. The Wb5 construct was the best amongst all the candidates, with no cross-reactivity with Loa- or Onchocerca-infected sera.

### Example 2

### Isotype Specificity and Screening Individual Samples with Wb5

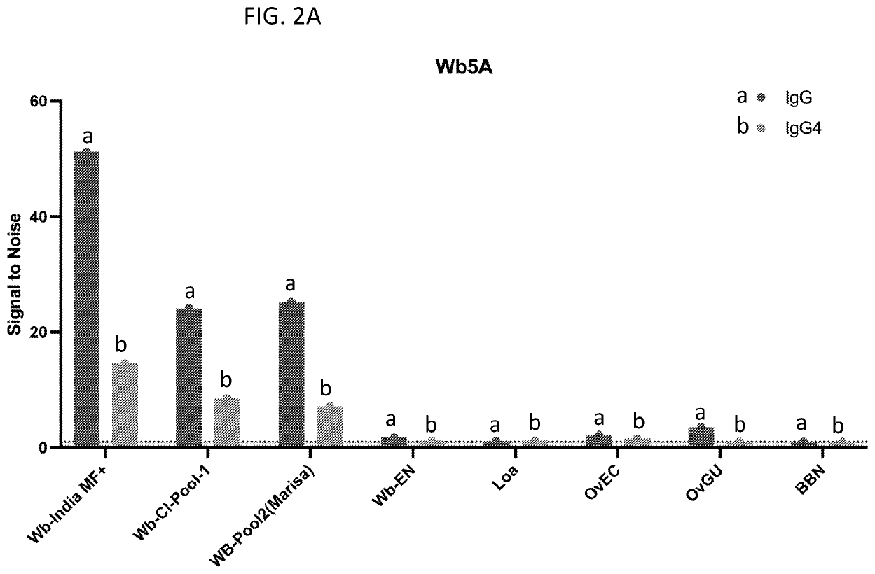

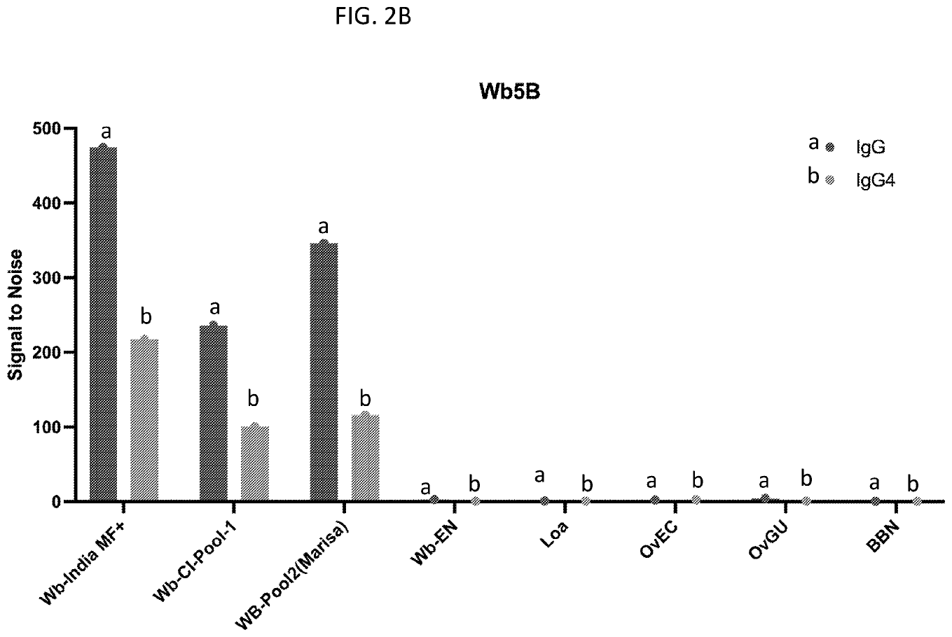

The sero-reactivity was checked at the level of total IgG and IgG4 for all the proteins. Wb5 was the best candidate based on reactivity (FIGS. 2A and 2B). Among the two different forms of Wb5, Wb5B (mature protein without signal sequence) had greater signal intensity compared to Wb5A (full-length protein including signal sequence). The screening by LIPS was as described in Example 1, with the additional screening using UltraLink BioSupport beads (ThermoFisher) covalently coupled with anti-human IgG4 (HP-6025) for assessing IgG4 reactivity.

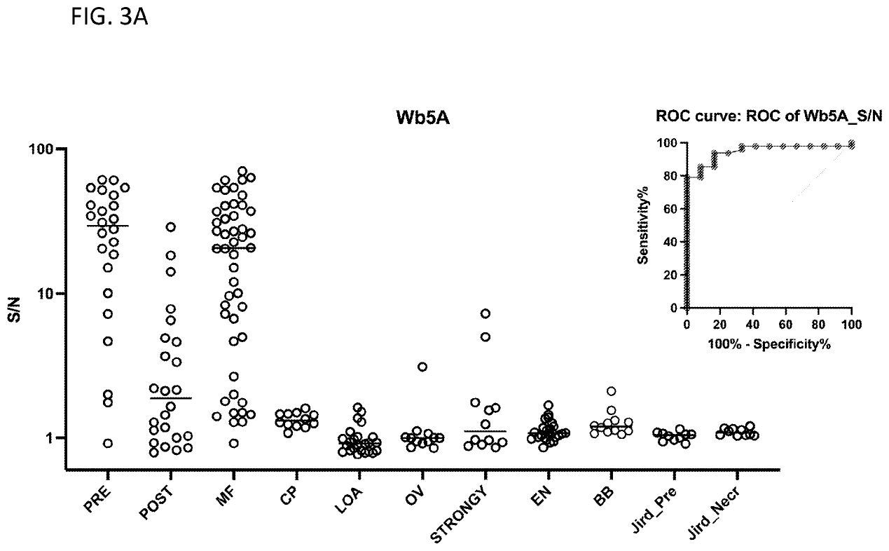

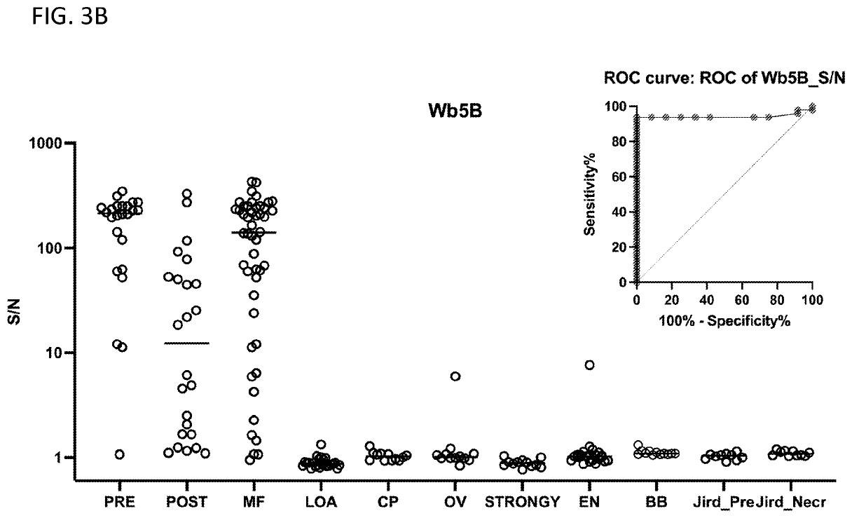

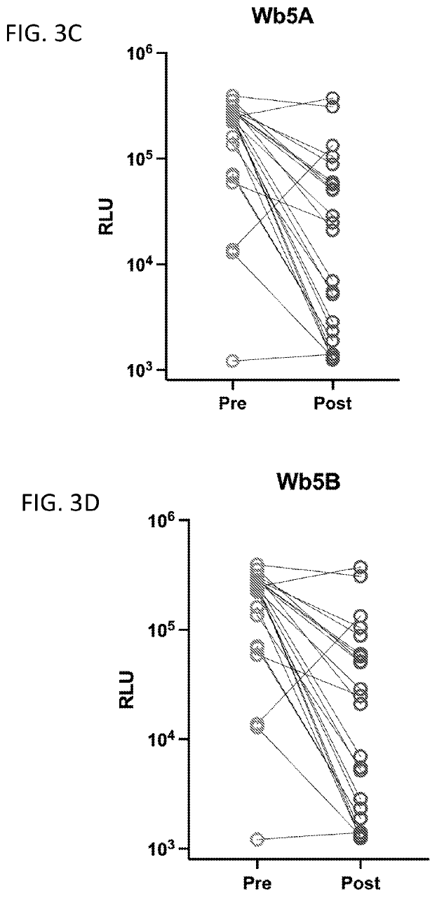

Wb5A and Wb5B fusion proteins were screened by LIPS assay (as described in Example 1) with individual sera from Wb-infected mt-positive sera (MF; PRE; POST), Wb-chronic pathology (CP; lymphedema), endemic normals from India (EN); healthy blood bank normals (BB), Loa-infected (LOA); Onchocerca-infected (OV); Strongyloides (STRONGY), or gerbil sera infected with B. malayi. Brugia-infected sera were not available, and hence used the gerbil infected sera as a surrogate; however, it is unknown if gerbils elicit antibodies to Wb5 or not. As shown in FIGS. 3A-3D, at specificity of 100% using blood bank normals, Wb5A and Wb5B had sensitivities of 79.17% and 93.75%. It is not clear if the two outlier samples—one each from endemic normal and Onchocerca—were contaminated or mis-labeled. Further, screening the Wb5A and Wb5B reactivity with sera from Wb-infected individuals from Cook Islands that have presumably cleared infection in 1992 (compared to their positive status in 1974) indicated that there is a drop in the IgG levels (FIG. 3D).

### Example 3

### Detection of Wb5 Using Luminex-Based Assays

A subset of Wb-infected and microfilariae positive samples (n=85) were tested in parallel on the LIPS platform using beads coupled to anti-human IgG4 and on the Luminex platform. The Luminex beads were coupled with the Wb5-Fc fusion protein (SEQ ID NO: 16).

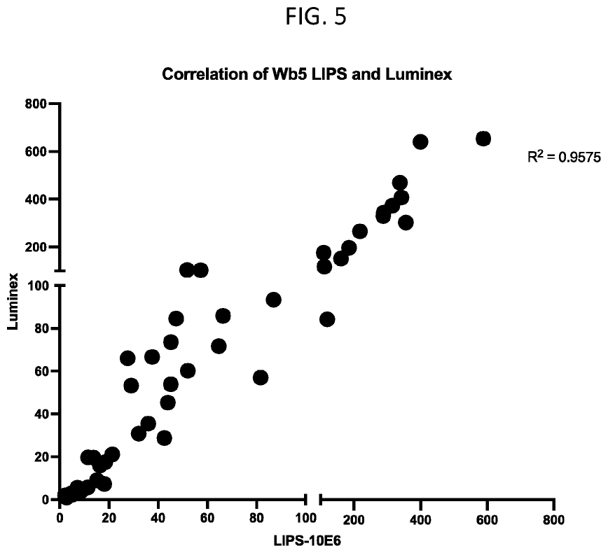

The ability of Wb5 to detect W. bancrofti microfilaria positive samples was highly correlated in LIPS and Luminex platforms. Detection by Wb5 of a subset of W. bancrofti microfilaria positive samples was tested in both LIPS and Luminex IgG4-based systems (n=85). Detection intensity was highly linearly correlated (FIG. 5; R^{2=0.9575}).

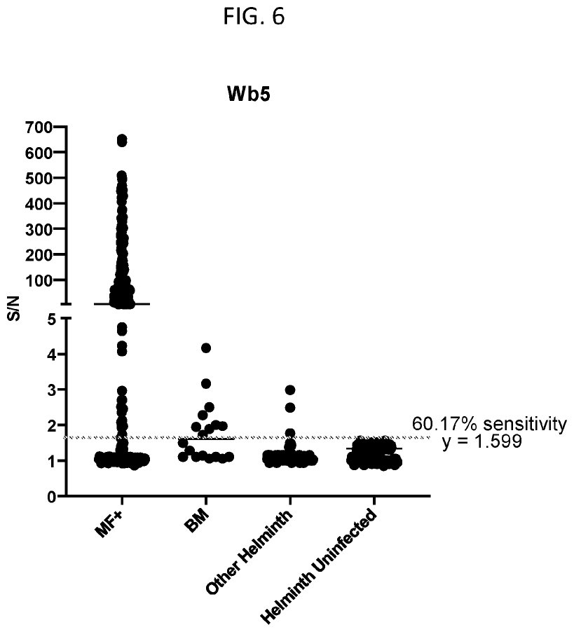

IgG4 reactivity to Wb5 in a Luminex-based assay was detected in individual sera from microfilaria positive individuals infected with W. bancrofti microfilaria positive, B. malayi, other helminths (O. volvulus, L. loa, S. stercoralis, M. perstans), or healthy blood bank and endemic normal (FIG. 6). This demonstrates that use of Wb5 in the Luminex platform had a sensitivity of about 60% in detecting W. bancrofti infection with 100% specificity (ruling out other filarial/helminth infections).

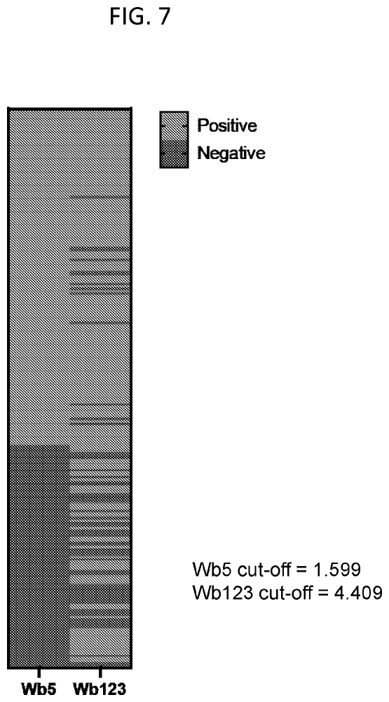

A heatmap depicting Wb5 and Wb123 positive and negative results from IgG4 Luminex testing was generated (FIG. 7). A total of 231 MF+ samples were tested, of which 174 were Wbl23 positive and 139 were Wb5 positive. There were 13 Wb5 positive/Wbl23 negative and 48 Wb5 negative/Wbl23 positive samples. Thus, testing of Wb5 in combination with Wb123 improves sensitivity of detection of W. bancrofti infection to 81%.

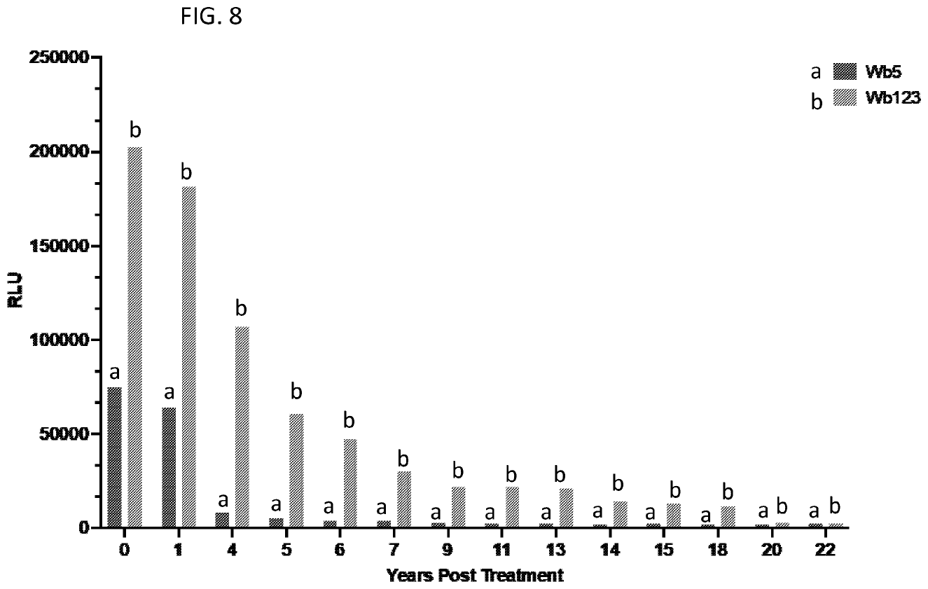

Titer kinetics of Wb5 and Wb123 were assessed by LIPS screening of IgG anti-Wb5 and anti-Wbl23 antibodies in a single patient over a 22-year time period following definitive filarial treatment. Anti-Wb5 IgG levels decreased more rapidly and earlier following treatment than for Wb123 (FIG. 8).

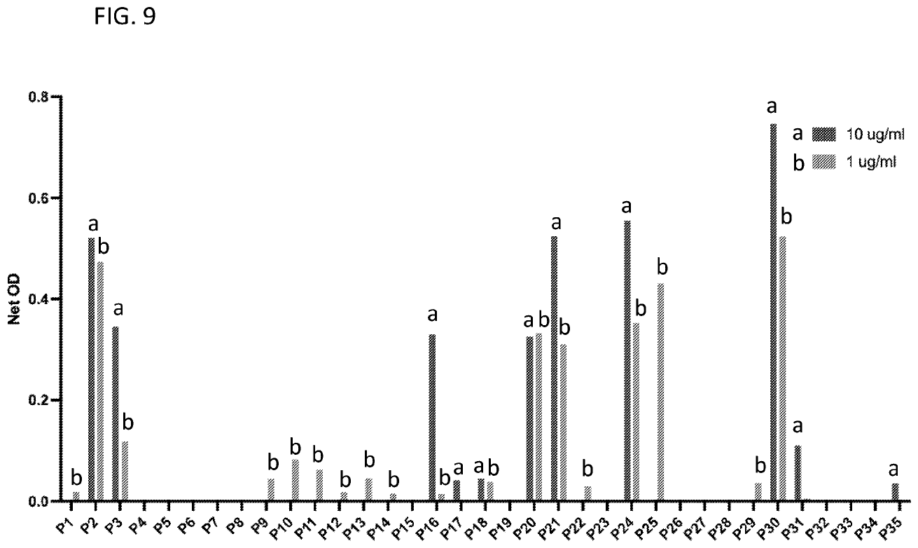

Overlapping peptides of Wb5 were synthesized and screened with pooled infected sera. The protein regions that span the P1-P2; P15-P17; P20-25; and P30-P31 regions were found to be immunogenic in nature (FIG. 9).

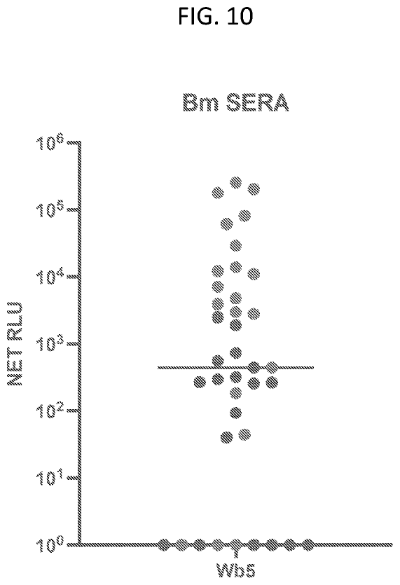

IgG4 reactivity was also assessed using Luminex assay with individuals infected with Brugia spp. (FIG. 10).

It will be apparent that the precise details of the methods or compositions described may be varied or modified without departing from the spirit of the described aspects of the disclosure. We claim all such modifications and variations that fall within the scope and spirit of the claims below.

1. A fusion protein comprising a Wuchereria bancrofti or Brugia sp. Wb5 protein or a portion thereof linked to a reporter protein or a tag.

2. The fusion protein of claim 1, wherein the Wb5 protein or portion thereof comprises:
an amino acid sequence with at least 95% sequence identity to the amino acid sequence of SEQ ID NO: 2, or a portion thereof;
an amino acid sequence with at least 95% sequence identity to the amino acid sequence of SEQ ID NO: 4, or a portion thereof;
an amino acid sequence with at least 95% sequence identity to the amino acid sequence of SEQ ID NO: 8, or a portion thereof;
an amino acid sequence with at least 95% sequence identity to the amino acid sequence of SEQ ID NO: 10, or a portion thereof; an amino acid sequence with at least 95% sequence identity to the amino acid sequence of any one of SEQ ID NOs: 18-21;
the amino acid sequence of SEQ ID NO: 2, or a portion thereof;
the amino acid sequence of SEQ ID NO: 4, or a portion thereof;
the amino acid sequence of SEQ ID NO: 8, or a portion thereof;
the amino acid sequence of SEQ ID NO: 10, or a portion thereof; or
the amino acid sequence of any one of SEQ ID NOs: 18-21.

3. (canceled)

4. The fusion protein of claim 1, wherein;
a) the reporter protein comprises luciferase, or
b) the tag is a 6× histidine tag, glutathione-S-transferase (GST), IgG-Fc, maltose-binding protein, FLAG-tag, or biotin.

5. The fusion protein of claim 4, wherein the luciferase is Renilla luciferase.

6. (canceled)

7. The fusion protein of claim 4, wherein the tag is capable of being cleaved from the Wb5 protein.

8. The fusion protein of claim 4, wherein the fusion protein comprises an amino acid sequence with at least 95% sequence identity to the amino acid sequence of SEQ ID NO: 14 or SEQ ID NO: 16, or the fusion protein comprises or consists of the amino acid sequence of SEQ ID NO: 14 or SEQ ID NO: 16.

9. (canceled)

10. A nucleic acid encoding the fusion protein of claim 1.

11. The nucleic acid of claim 10, wherein the nucleic acid has at least 95% sequence identity to the nucleic acid sequence of SEQ ID NO: 15 or SEQ ID NO: 17, or comprises or consists of SEQ ID NO: 15 or SEQ ID NO: 17.

12. (canceled)

13. A codon-optimized nucleic acid encoding a Wuchereria bancrofti or Brugia sp. Wb5 protein or a portion thereof.

14. The codon-optimized nucleic acid of claim 13, wherein:
the nucleic acid encodes a Wb5 protein with at least 95% sequence identity to the amino acid sequence of SEQ ID NO: 2, or a portion thereof;
the nucleic acid encodes a Wb5 protein with at least 95% sequence identity to the amino acid sequence of SEQ ID NO: 4, or a portion thereof;
the nucleic acid encodes a Wb5 protein with at least 95% sequence identity to the amino acid sequence of SEQ ID NO: 8, or a portion thereof;
the nucleic acid encodes a Wb5 protein with at least 95% sequence identity to the amino acid sequence of SEQ ID NO: 10, or a portion thereof;
the nucleic acid encodes a Wb5 protein with at least 95% sequence identity to the amino acid sequence of any one of SEQ ID NOs: 18-21;
the nucleic acid encodes a Wb5 protein comprising or consisting of the amino acid sequence of SEQ ID NO: 2, or a portion thereof;
the nucleic acid encodes a Wb5 protein comprising or consisting of the amino acid sequence of SEQ ID NO: 4, or a portion thereof;
the nucleic acid encodes a Wb5 protein comprising or consisting of the amino acid sequence of SEQ ID NO: 8, or a portion thereof;
the nucleic acid encodes a Wb5 protein comprising or consisting of the amino acid sequence of SEQ ID NO: 10, or a portion thereof; or
the nucleic acid encodes a Wb5 protein comprising or consisting of the amino acid sequence of any one of SEQ ID NOs: 18-21.

15. (canceled)

16. The codon-optimized nucleic acid of claim 13, wherein:
the nucleic acid has at least 95% sequence identity to the nucleic acid sequence of SEQ ID NO: 1, or a portion thereof;
the nucleic acid has at least 95% sequence identity to the nucleic acid sequence of SEQ ID NO: 3, or a portion thereof;
the nucleic acid has at least 95% sequence identity to the nucleic acid sequence of SEQ ID NO: 11, or a portion thereof;
the nucleic acid has at least 95% sequence identity to the nucleic acid sequence of SEQ ID NO: 12, or a portion thereof;
the nucleic acid has at least 95% sequence identity to the nucleic acid sequence of SEQ ID NO: 13, or a portion thereof;
the nucleic acid comprises or consists of SEQ ID NO: 1, or a portion thereof;
the nucleic acid comprises or consists of SEQ ID NO: 3, or a portion thereof;
the nucleic acid comprises or consists of SEQ ID NO: 11, or a portion thereof;
the nucleic acid comprises or consists of SEQ ID NO: 12, or a portion thereof; or
the nucleic acid comprises or consists of SEQ ID NO: 13, or a portion thereof.

17. (canceled)

18. A vector comprising the nucleic acid of claim 10.

19. (canceled)

20. A host cell comprising the nucleic acid of claim 10.

21. The host cell of claim 20, wherein the host cell is a bacterial, insect, or mammalian cell.

22. The host cell of claim 21, wherein the host cell is an HEK293 cell.

23. (canceled)

24. A method of detecting presence of antibodies to Wuchereria bancrofti or Brugia sp. in a sample, comprising:
contacting the sample with the fusion protein of claim 1 under conditions sufficient to form a complex between the Wb5 protein or a portion thereof and an antibody to W. bancrofti or Brugia sp.;
contacting the complex with an immobilized binding agent capable of binding to the antibody to W. bancrofti or Brugia sp., thereby forming an immobilized complex comprising the fusion protein; and
detecting output from the reporter protein in the immobilized complex, thereby detecting presence of antibodies to W. bancrofti or Brugia sp. in the sample.

25. The method of claim 24, wherein:
a) the immobilized binding agent comprises protein A, protein G, or protein A/G; and/or
b) the reporter protein comprises luciferase.

26. (canceled)

27. The method of claim 25, further comprising contacting the immobilized complex comprising the fusion protein with a luciferase enzyme substrate.

28. The method of claim 24, wherein detecting output from the reporter protein comprises adding a reporter domain substrate to the immobilized complex.

29. A method of detecting presence of antibodies to Wuchereria bancrofti or Brugia sp. in a sample, comprising an immunoassay detecting presence of antibodies that specifically bind to a Wb5 protein, or a portion thereof.

30. The method of claim 29, wherein the immunoassay is an ELISA assay or lateral flow assay.

31. (canceled)

32. The method of claim 29, wherein the Wb5 protein comprises:
a protein with a least 95% sequence identity to the amino acid sequence of SEQ ID NO: 2, or a portion thereof;
a protein with a least 95% sequence identity to the amino acid sequence of SEQ ID NO: 4, or a portion thereof;
a protein with a least 95% sequence identity to the amino acid sequence of SEQ ID NO: 8, or a portion thereof;
a protein with a least 95% sequence identity to the amino acid sequence of SEQ ID NO: 10, or a portion thereof;
a protein comprising at least 95% sequence identity to the amino acid sequence of any one of SEQ ID NOs: 18-21;
SEQ ID NO: 2 or a portion thereof;
SEQ ID NO: 4 or a portion thereof;
SEQ ID NO: 8 or a portion thereof;
SEQ ID NO: 10 or a portion thereof; or
any one of SEQ ID NOs: 18-21.

33. (canceled)

34. The method of claim 29, wherein the Wb5 protein or portion thereof is bound to a solid support.

35. The method of claim 29, wherein the method comprises:
contacting the Wb5 protein or portion thereof with the sample to form a first complex comprising the Wb5 protein and an antibody to W. bancrofti or Brugia sp.;
contacting the complex with a secondary antibody comprising a detectable label to form a second complex comprising the first complex and the secondary antibody; and
detecting the presence of the second complex by detecting output from the detectable label.

36. The method of claim 35, wherein the secondary antibody is an anti-human IgG antibody.

37. (canceled)

38. The method of claim 24, further comprising detecting presence of antibodies to W. bancrofti Wb123 protein in the sample.

39. The method of claim 24, wherein the sample comprises:
a) a sample from at least one subject infected with or suspected of being infected with W. bancrofti or Brugia sp.;
b) a pooled sample from a plurality of subjects; and/or
c) blood or serum.

40-41. (canceled)

42. The method of claim 24, wherein the subject is human.

43. The method of claim 39, further comprising diagnosing the subject or plurality of subjects with active lymphatic filariasis when presence of antibodies to the Wb5 protein are detected in the sample.

44. The method of claim 43, further comprising treating the subject for lymphatic filariasis or implementing one or more lymphatic filariasis control regimens in a population from which the pooled sample was obtained.

45-46. (canceled)

47. A kit for detecting antibodies to Wuchereria bancrofti or Brugia sp., comprising:
the fusion protein of claim 1; and
an immobilized binding agent capable of binding to the antibody to W. bancrofti or Brugia sp.

48-50. (canceled)


SEQUENCE LISTING (23 sequences)

SEQ ID NO: 1 - DNA, 465 bp, synthetic construct
  source 1..465: mol_type = other DNA
ggatccatgcggtccgcccagttccccttctttatctctcctctgctgttctttatcatcggcaccctggccctgctggccatgtctcagaggtgcgcccctagcatcaacaaggacaacctgaatcacgaggatggcgacgatggcaacatcaacaataacggcgacaataacatcaatggcgacgataacaacatcaacaacaacatcaacgacaacaataacttcctgcagcagcagcggtacgatgaggccctgagcatcgagggcctgtcctgggacgatatcaccgaggaggagagggatatcctgatgagcctgctgctgaaccgctatatcaatgcctccatgctgccatggaataacaatggcatccccgtggtggtgaacgtgatccggtctgccctgccccacaatagaggccagtttatcggctacacaggcctgctggagctgtgactcgag

SEQ ID NO: 2 - protein, 123 aa, Wuchereria bancrofti
QRCAPSINKDNLNHEDGDDGNINNNGDNNINGDDNNINNNINDNNNFLQQQRYDEALSIEGLSWDDITEEERDILMSLLLNRYINASMLPWNNNGIPVVVNVIRSALPHNRGQFIGYTGLLEL

SEQ ID NO: 3 - DNA, 450 bp, synthetic construct
  source 1..450: mol_type = other DNA
atgcggtccgcccagttccccttctttatctctcctctgctgttctttatcatcggcaccctggccctgctggccatgtctcagaggtgcgcccctagcatcaacaaggacaacctgaatcacgaggatggcgacgatggcaacatcaacaataacggcgacaataacatcaatggcgacgataacaacatcaacaacaacatcaacgacaacaataacttcctgcagcagcagcggtacgatgaggccctgagcatcgagggcctgtcctgggacgatatcaccgaggaggagagggatatcctgatgagcctgctgctgaaccgctatatcaatgcctccatgctgccatggaataacaatggcatccccgtggtggtgaacgtgatccggtctgccctgccccacaatagaggccagtttatcggctacacaggcctgctggagctg

SEQ ID NO: 4 - protein, 150 aa, Wuchereria bancrofti
MRSAQFPFFISPLLFFIIGTLALLAMSQRCAPSINKDNLNHEDGDDGNINNNGDNNINGDDNNINNNINDNNNFLQQQRYDEALSIEGLSWDDITEEERDILMSLLLNRYINASMLPWNNNGIPVVVNVIRSALPHNRGQFIGYTGLLEL

SEQ ID NO: 5 - DNA, 450 bp, Wuchereria bancrofti
  source 1..450: mol_type = unassigned DNA
atgagaagcgcacaatttccattcttcatatcaccattactttttttcattatcggtacactagcattattagcaatgtcacaaagatgtgctccgagcattaacaaagacaacttgaaccatgaagatggtgatgacggtaacataaacaataatggtgacaataatattaatggtgacgacaacaacattaacaacaacattaatgacaacaataattttctccagcaacaaagatatgatgaagctctttccattgaaggcttatcttgggatgatatcacggaagaggaaagagatattctcatgtcattgctacttaaccgttatattaatgcatcaatgttaccgtggaataataatggtatacctgttgtggtcaatgttattagaagtgccttacctcataatcgtggtcaatttatcggctatactggcctgttggaactt

SEQ ID NO: 6 - DNA, 213 bp, Wuchereria bancrofti
  source 1..213: mol_type = unassigned DNA
atgagaagcgcacaatttccattcttcatatcaccattactttttttcattatcggtacactagcattattagcaatgtcacaaagattaataatggtatacctgttgtggtcaatgttattagaagtgccttacctcataatcgtggtcaatttatcggctatactggcctgttggaactttaagcttatttatgcgcacatattaatacca

SEQ ID NO: 7 - DNA, 550 bp, Wuchereria bancrofti
  source 1..550: mol_type = unassigned DNA
ttaaggtatcgaattccggaacgatgagaagcgcacaatttccattcttcatatcaccattactttttttcattatcggtacactagcattattagcaatgtcacaaagatgtgctccgagcattaacaaagacaacttgaaccatgaagatggtgatgacggtaacataaacaataatggtgacaataatattaatggtgacgacaacaacattaacaacaacattaatgacaacaataattttctccagcaacaaagatatgatgaagctctttccattgaaggcttatcttgggatgatatcacggaagaggaaagagatattctcatgtcattgctacttaaccgttatattaatgcatcaatgttaccgtggaataataatggtatacctgttgtggtcaatgttattagaagtgccttacctcataatcgtggtcaatttatcggctatactggcctgttggaactttaagcttatttatgcgcacatattaataccataaaatttggtgatatttattgttgaataaaattgaaaaactgaaa

SEQ ID NO: 8 - protein, 71 aa, Wuchereria bancrofti
MRSAQFPFFISPLLFFIIGTLALLAMSQRLIMVYLLWSMLLEVPYLIIVVNLSAILACWNFKLIYAHILIP

SEQ ID NO: 9 - DNA, 446 bp, Brugia malayi
  source 1..446: mol_type = unassigned DNA
atgagaagcgcacaatttccacttttcatatcaccattatctttttgcattatcggtgcactagcattactagcaatgtcgcaaagatgtgctccgagcattaacagaaataacttaaatcatgaagatggtgatgacggtaacataaacgataatggtgacaataatgttaatggtgacgatggcaatatcaacaacgttaatgacaacaataattttctccagcagcaaagatatgatgaagctctttccattgaaggcttatcttgggataatattacggaagaggagagagatattcttatgtcattgctacttaaccgatatattaatgcatcaatgctaccgtggaataatgatggtatacctgttgtggtcaatgttattagaagtgctttacctcgcaattatgccaatttatcggctatactggcctattggagcat

SEQ ID NO: 10 - protein, 149 aa, Brugia malayi
MRSAQFPLFISPLSFCIIGALALLAMSQRCAPSINRNNLNHEDGDDGNINDNGDNNVNGDDGNINNVNDNNNFLQQQRYDEALSIEGLSWDNITEEERDILMSLLLNRYINASMLPWNNDGIPVVVNVIRSALPRNYGQFIGYTGLLEH

SEQ ID NO: 11 - DNA, 453 bp, synthetic construct
  source 1..453: mol_type = other DNA
atgcgaagtgcacaatttccattttttatctctcctctgctgttcttcatcatcggcaccctggctcagagatgcgccccatctatcaacaaggacaacctgaaccacgaggacggcgatgatggcaacatcaacaacaacggagacaataatattaatggagacgacaacaacatcaacaacaacatcaacgacaacaacaacttcctgcagcagcaacgctacgacgaggccctgtccatcgagggcctgtcttgggacgatatcaccgaggaagagcgggacatcctgatgtccctgctgctgaacagatacatcaacgcctccatgctgccttggaacaataacggcatccccgtcgtggtgaacgtgatcagaagcgctctgcctcacaaccggggccagtttatcggctacaccggcctcctggaactgcaccaccatcaccaccac

SEQ ID NO: 12 - DNA, 372 bp, synthetic construct
  source 1..372: mol_type = other DNA
atgcaaaggtgtgctccctcaataaataaagacaacctgaatcatgaggatggcgacgacggcaatatcaacaacaacggcgacaacaacatcaatggcgatgataacaacatcaacaacaacatcaatgataataacaacttcctgcagcaacaacgttacgacgaggcgctgtctattgaaggtctctcctgggatgacatcaccgaagaggagcgcgatatcctgatgagcctgttattgaatcgctacattaacgctagcatgctgccgtggaataacaacggtattccggtggttgttaatgtgattcgtagcgcactgccgcataatcgtggtcagtttattggttatacgggtttgttggaactg

SEQ ID NO: 13 - DNA, 402 bp, synthetic construct
  source 1..402: mol_type = other DNA
gccgccaccatgcagcggtgcgcaccttcgataaacaaagataacctgaatcatgaggatggtgatgacgggaacataaacaataacggcgacaataacataaatggcgacgacaacaatatcaacaacaatatcaacgacaataataactttttgcagcaacaacgttacgatgaagcccttagtattgaggggctatcctgggatgacattaccgaagaagagagggatatactaatgtcattattgctcaaccgctatattaatgcgagcatgctgccatggaataataacggcatccccgtagtcgttaatgtgattcgatctgctttaccgcataatagaggacagttcatcggttatactggactccttgaattacatcaccatcaccaccactga

SEQ ID NO: 14 - protein, 158 aa, synthetic construct
MRSAQFPFFISPLLFFIIGTLAHHHHHHENLYFQGQRCAPSINKDNLNHEDGDDGNINNNGDNNINGDDNNINNNINDNNNFLQQQRYDEALSIEGLSWDDITEEERDILMSLLLNRYINASMLPWNNNGIPVVVNVIRSALPHNRGQFIGYTGLLEL

SEQ ID NO: 15 - DNA, 474 bp, synthetic construct
  source 1..474: mol_type = other DNA
atgcgaagtgcacaatttccattttttatctctcctctgctgttcttcatcatcggcaccctggctcaccaccaccaccaccacgagaacctgtacttccagggccagagatgcgccccatctatcaacaaggacaacctgaaccacgaggacggcgatgatggcaacatcaacaacaacggagacaataatattaatggagacgacaacaacatcaacaacaacatcaacgacaacaacaacttcctgcagcagcaacgctacgacgaggccctgtccatcgagggcctgtcttgggacgatatcaccgaggaagagcgggacatcctgatgtccctgctgctgaacagatacatcaacgcctccatgctgccttggaacaataacggcatccccgtcgtggtgaacgtgatcagaagcgctctgcctcacaaccggggccagtttatcggctacaccggcctcctggaactg

SEQ ID NO: 16 - protein, 413 aa, synthetic construct
MRSAQFPFFISPLLFFIIGTLADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKAAANSSIDLISVPVDSRRPACKIPNDLKQKVMNHDYKDDDDKRCAPSINKDNLNHEDGDDGNINNNGDNNINGDDNNINNNINDNNNFLQQQRYDEALSIEGLSWDDITEEERDILMSLLLNRYINASMLPWNNNGIPVVVNVIRSALPHNRGQFIGYTGLLEL

SEQ ID NO: 17 - DNA, 1239 bp, synthetic construct
  source 1..1239: mol_type = other DNA
atgcgaagtgcacaatttccattttttatctctcctctgctgttcttcatcatcggcaccctggctgacaagacccacacctgccccccctgccccgcccccgagctgctgggcggcccctccgtgttcctgttcccccccaagcccaaggacaccctgatgatctccaggacccccgaggtgacctgcgtggtggtggacgtgtcccacgaggaccccgaggtgaagttcaactggtacgtggacggcgtggaggtgcacaacgccaagaccaagcccagggaggagcagtacaactccacctacagggtggtgtccgtgctgaccgtgctgcaccaggactggctgaacggcaaggagtacaagtgcaaggtgtccaacaaggccctgcccgcccccatcgagaagaccatctccaaggccaagggccagcccagggagccccaggtgtacaccctgcccccctccagggaggagatgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtccaggtggcagcagggcaacgtgttctcctgctccgtgatgcacgaggccctgcacaaccactacacccagaagtccctgtccctgtcccccggcaaggccgccgccaactcctccatcgacctgatctccgtgcccgtggactccaggaggcccgcctgcaagatccccaacgacctgaagcagaaggtgatgaaccacgactacaaggacgacgacgacaagagatgcgccccatctatcaacaaggacaacctgaaccacgaggacggcgatgatggcaacatcaacaacaacggagacaataatattaatggagacgacaacaacatcaacaacaacatcaacgacaacaacaacttcctgcagcagcaacgctacgacgaggccctgtccatcgagggcctgtcttgggacgatatcaccgaggaagagcgggacatcctgatgtccctgctgctgaacagatacatcaacgcctccatgctgccttggaacaataacggcatccccgtcgtggtgaacgtgatcagaagcgctctgcctcacaaccggggccagtttatcggctacaccggcctcctggaactg

SEQ ID NO: 18 - protein, 19 aa, synthetic construct
QFPFFISPLLFFIIGTLAL

SEQ ID NO: 19 - protein, 15 aa, synthetic construct
INGDDNNINNNINDN

SEQ ID NO: 20 - protein, 35 aa, synthetic construct
QQRYDEALSIEGLSWDDITEEERDILMSLLLNRYI

SEQ ID NO: 21 - protein, 19 aa, synthetic construct
PWNNNGIPVVVNVIRSALP

SEQ ID NO: 22 - protein, 7 aa, synthetic construct
ENLYFQG

SEQ ID NO: 23 - protein, 8 aa, synthetic construct
DYKDDDDK